(19) United States
(12) Patent Application Publication
(10) Pub. No.: US 2026/0306190 A1
(43) Pub. Date: Oct. 8, 2026

(54) SYSTEMS AND METHODS FOR INTRAOPERATIVELY UPDATING A GRAPHICAL USER INTERFACE BASED ON INTRAOPERATIVE IMAGING DATA

(71) Applicant: INTUITIVE SURGICAL OPERATIONS, INC., Sunnyvale, CA (US)

(72) Inventors: Zachary Moller, San Francisco, CA (US); Troy K. Adebar, San Jose, CA (US); Joy Janku, Amsterdam (NL); Gavin Jensen, Saratoga Springs, UT (US); Bai Wang, Palo Alto, CA (US)

(21) Appl. No.: 19/482,138

(22) PCT Filed: May 7, 2024
(86) PCT No.: PCT/US2024/028100

§ 371 Date: Nov. 6, 2025

Related U.S. Application Data
(60) Provisional application No. 63/500,702, filed on May 8, 2023.

(51) Int. Cl. A61B 34/00; A61B 34/10; A61B 34/20; A61B 34/35; G06T 7/11; G06T 7/33

(52) U.S. Cl. CPC A61B 34/25; A61B 34/35; G06T 7/11; G06T 7/337; A61B 2034/107; A61B 2034/2065; G06T 2200/24; G06T 2207/30004

(57) ABSTRACT

A system comprises a display system, an elongate device, a medical instrument, and a control system. The control system is configured to display, in a graphical user interface (GUI) on the display system, intraoperative imaging data of a patient anatomy received from an external imaging system; display a graphical indicator overlaid on the intraoperative imaging data; register the intraoperative imaging data to the elongate device; based on the registration, transform the graphical indicator from an intraoperative imaging data reference frame to an elongate device reference frame; and based on a target position of a distal tip of the medical instrument displayed via the graphical indicator, provide navigation guidance to guide the elongate device to a target location in the patient anatomy. When the elongate device is positioned at the target location, the target position of the distal tip of the medical instrument is within a deployment range of the instrument.

### CROSS-REFERENCED APPLICATIONS

This application claims priority to and the benefit of U.S. Provisional Applications No. 63/500,702, filed May 8, 2023, and entitled “Systems and Methods for Intraoperatively Updating a Graphical User Interface Based on Intraoperative Imaging Data,” which is incorporated by reference herein in its entirety.

### FIELD

Examples described herein relate to systems and methods for planning and performing an image-guided procedure, such as systems and methods for planning a treatment zone and intraoperatively confirming the treatment zone.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, an operator may insert minimally invasive medical tools to reach a target tissue location. Minimally invasive medical tools include instruments such as therapeutic, diagnostic, biopsy, and surgical instruments. Medical tools may be inserted into anatomical passageways and navigated toward a region of interest within a patient anatomy. Navigation may be assisted using images of the anatomical passageways, obtained preoperatively and/or intraoperatively. Improved systems and methods are needed to enhance information provided to a user via a graphical user interface (GUI) based on information received from intraoperative imaging.

### SUMMARY

Various features may improve navigation guidance that is provided to guide an elongate device to a target location in a patient anatomy. The following presents a simplified summary of various examples described herein and is not intended to identify key or critical elements or to delineate the scope of the claims.

Consistent with some examples, a system is provided. The system includes a display system, an elongate device, a medical instrument configured to extend within the elongate device and a control system communicatively coupled to the display system. The control system is configured to display intraoperative imaging data of a patient anatomy received from an external imaging system. The intraoperative imaging data is displayed in a graphical user interface (GUI) on the display system. The control system is further configured to display a graphical indicator overlaid on the intraoperative imaging data. The graphical indicator indicates a target position of a distal tip of the medical instrument in the patient anatomy. The control system is further configured to register the intraoperative imaging data to the elongate device. The control system is further configured to, based on the registration, transform the graphical indicator from a reference frame of the intraoperative imaging data to a reference frame of the elongate device. The control system is further configured to, based on the target position of the distal tip of the medical instrument displayed via the graphical indicator, provide navigation guidance to guide the elongate device to a target location in the patient anatomy. When the elongate device is positioned at the target location, the target position of the distal tip of the medical instrument is within a deployment range of the medical instrument.

Consistent with other examples, a system is provided. The system includes a display system, an elongate device, a medical instrument configured to extend within the elongate device, and a control system. The control system is configured to display intraoperative imaging data of a patient anatomy received from an external imaging system. The intraoperative imaging data is displayed in a graphical user interface (GUI) on the display system. The control system is further configured to display treatment information overlaid on the intraoperative imaging data. The treatment information illustrates one or more treatment parameters for treating an anatomical target in the patient anatomy. The control system is further configured to register the intraoperative imaging data to the elongate device. The control system is further configured to, based on the registration, transform the treatment information from a reference frame of the intraoperative imaging data to a reference frame of the elongate device. The control system is further configured to, based on the treatment information, provide navigation guidance to guide the elongate device to a target location in the patient anatomy.

Consistent with other examples, a method is provided. The method includes displaying intraoperative imaging data of a patient anatomy received from an external imaging system. The intraoperative imaging data is displayed in a graphical user interface (GUI) on a display system. The method further includes displaying a graphical indicator overlaid on the intraoperative imaging data. The graphical indicator indicates a target position of a distal tip of a medical instrument in the patient anatomy. The medical instrument is configured to extend within an elongate device. The method further includes registering the intraoperative imaging data to the elongate device. The method further includes, based on the registration, transforming the graphical indicator from a reference frame of the intraoperative imaging data to a reference frame of the elongate device. The method further includes, based on the target position of the distal tip of the medical instrument displayed via the graphical indicator, providing navigation guidance to guide the elongate device to a target location in the patient anatomy. When the elongate device is positioned at the target location, the target position of the distal tip of the medical instrument is within a deployment range of the medical instrument.

Other examples include corresponding computer systems, apparatus, and computer programs recorded on one or more computer storage devices, each configured to perform the actions of any one or more methods described below.

It is to be understood that both the foregoing general description and the following detailed description are illustrative and explanatory in nature and are intended to provide an understanding of the various examples described herein without limiting the scope of the various examples described herein. In that regard, additional aspects, features, and advantages of the various examples described herein will be apparent to one skilled in the art from the following detailed description.

### BRIEF DESCRIPTIONS OF THE DRAWINGS

FIG. 1 illustrates a display system displaying an image of an elongate device registered to an anatomic model according to some examples.

FIG. 2 illustrates a method for updating a graphical user interface based on intraoperative image data according to some examples.

FIG. 3 illustrates a simplified diagram of a graphical user interface displaying image data from an intraoperative imaging procedure according to some examples.

FIG. 4 illustrates a method of registering image data to shape data from an instrument to update a location of a target in a model according to some examples.

FIGS. 5A-5E illustrate information displayed on a graphical user interface during various processes of FIG. 2 according to some examples.

FIGS. 6A-6B illustrate information displayed on a graphical user interface during navigation of an elongate device to an anatomical target according to some examples.

FIG. 7A illustrates information displayed on a graphical user interface based on intraoperative imaging data according to some examples.

FIG. 7B illustrates graphical information overlaid on intraoperative image data that is displayed in a graphical user interface according to some examples.

FIGS. 7C-7D illustrate graphical information overlaid on intraoperative image data that is displayed in a graphical user interface during updating of a location of a graphical indicator according to some examples.

FIG. 7E illustrates navigation guidance information displayed in a graphical user interface based on intraoperative image data and the graphical information overlaid on the intraoperative image data from FIG. 7B according to some examples.

FIGS. 7F-7I illustrate graphical information overlaid on intraoperative image data that is displayed in a graphical user interface according to some examples.

FIG. 8A illustrates a graphical user interface displaying intraoperative image data of an elongate device and a medical instrument according to some examples.

FIGS. 8B-8C illustrate a graphical user interface for adjusting a position of a medical instrument according to some examples.

FIG. 9 illustrates a simplified diagram of a robotic or teleoperated medical system according to some examples according to some examples.

FIG. 10 illustrates a simplified diagram of a medical instrument system and an intraoperative imaging system according to some examples according to some examples.

Various examples described herein and their advantages are described in the detailed description that follows. It should be appreciated that like reference numerals are used to identify like elements illustrated in one or more of the figures for purposes of illustrating but not limiting the various examples described herein.

### DETAILED DESCRIPTION

The techniques disclosed in this document may be used to provide and update information provided to a user via a graphical user interface during minimally invasive procedures using intraoperative imaging, such as cone beam computerized tomography (CT) imaging. In some examples, a model of an anatomical structure may be constructed based upon a preoperative imaging procedure and displayed on a graphical user interface (GUI). The model may be revised based upon an intraoperative imaging procedure performed during a minimally invasive procedure. In some examples, the image data produced by the intraoperative imaging may be utilized to revise a location of an instrument, an anatomic structure, or a target in the model constructed from a preoperative imaging procedure. In some examples, treatment information related to the minimally invasive procedure may be overlaid on intraoperative imaging data captured by the intraoperative imaging system and displayed on the GUI.

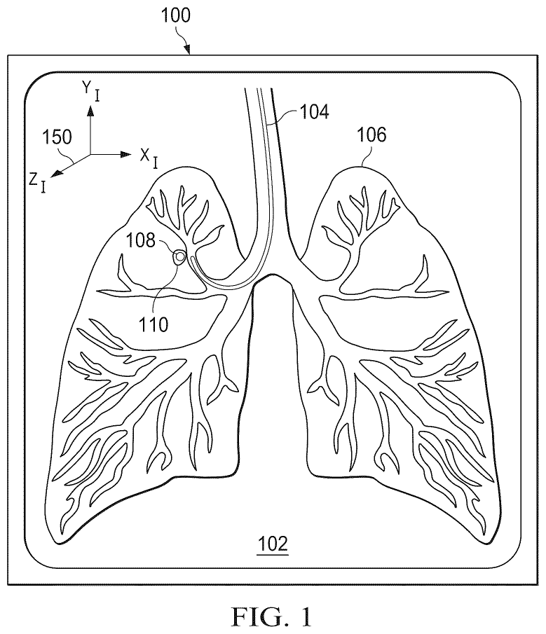

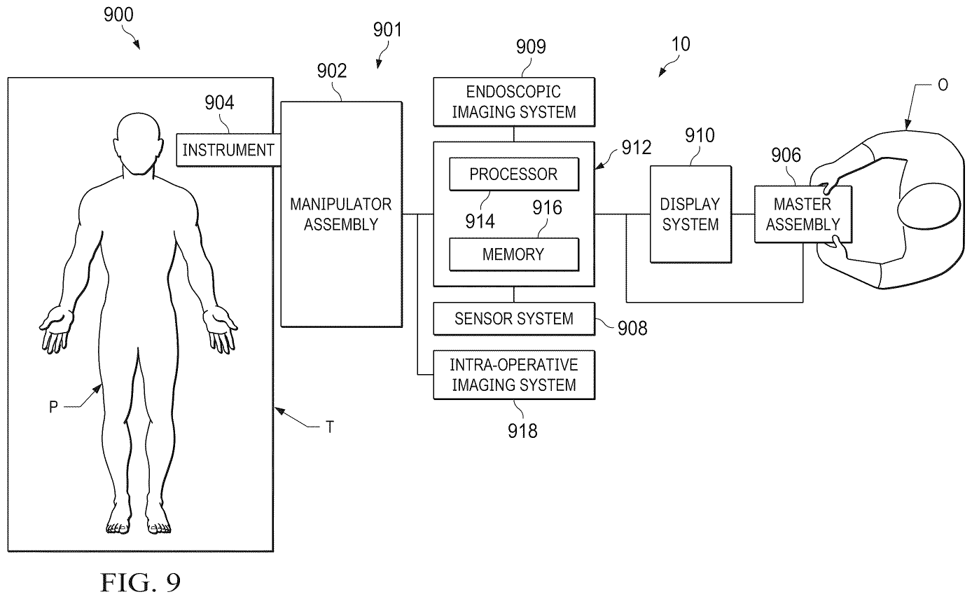

With reference to FIG. 1, an image-guided medical procedure, which may be manually performed, robot-assisted, or otherwise teleoperated, may be conducted in which a display system 100 may display a virtual navigation image 102, which may be referred to as a virtual navigation view. The virtual navigation image 102 includes an image reference frame (X_{I}, Y_{I}, Z_{I}) 150. An elongate device 104, such as a medical instrument (e.g., a catheter, a tool, a probe, an imaging device, etc.), may be registered (e.g., dynamically referenced) with an anatomic model 106 of a patient derived from preoperative image data obtained, for example, from a CT scan. The elongate device 104 may be a flexible, elongate device. In some examples, the elongate device 104 may be a catheter, a tool, a probe, an imaging device, etc. In some examples, the elongate device 104 may include one or more of a catheter, a tool, a probe, an imaging device, etc. The anatomic model 106 may include a target 108, such as a lesion or nodule of interest, with a margin 110 surrounding the target 108. In some examples, the virtual navigation image 102 may also or alternatively present a physician with a virtual image of the internal surgical site from a viewpoint of the elongate device 104, such as from a distal tip of the elongate device 104. In some examples, the display system 100 may also or alternatively present a real-time view from the distal tip of the elongate device 104, such as when the elongate device 104 includes an endoscope. In some examples, the elongate device 104 may be manipulated by a robot-assisted manipulator controlled by a control system (e.g., the control system 912 in FIG. 9), or processing system, which includes one or more processors. An example of a robot-assisted medical system will be described further at FIG. 9. In some examples, an ablation probe may extend through a lumen of the elongate device 104. In some examples, an ablation probe is the elongate device 104.

Generating the virtual navigation image 102 involves the registration of the image reference frame (X_{I}, Y_{I}, Z_{I}) 150 to a surgical reference frame (X_{S}, Y_{S}, Z_{S}) of the anatomy and/or an elongate device reference frame (_{XM,} Y_{M}, Z_{M}) of the elongate device 104. This registration may rotate, translate, or otherwise manipulate by rigid or non-rigid transforms points associated with the segmented instrument shape from the image data and/or points associated with the shape data from a shape sensor disposed along a length of the elongate device 104. This registration between the image reference frame and the elongate device reference frame may be achieved, for example, by using a point-based iterative closest point (ICP) technique as described in U.S. Provisional Pat. App. No. 62/205,440, filed on Aug. 14, 2015, entitled “Systems and Methods of Registration for Image-Guided Surgery” and in U.S. Provisional Pat. App. No. 62/205,433, filed on Aug. 14, 2015, entitled “Systems and Methods of Registration for Image-Guided Surgery,” which are incorporated by reference herein in their entireties. The registration may be achieved additionally or alternatively by another point cloud registration technique.

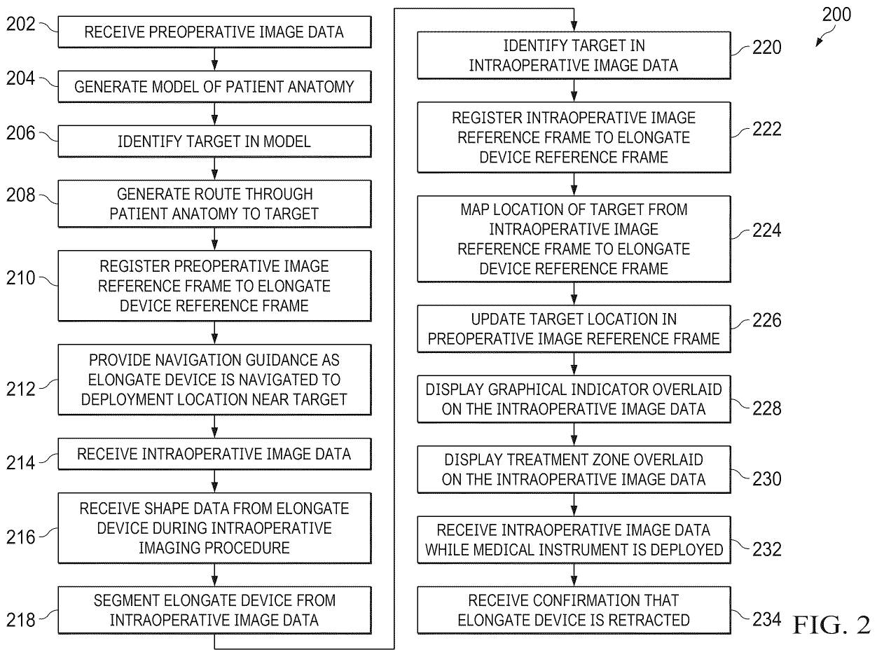

FIG. 2 illustrates an example of a method 200 for updating a graphical user interface (GUI) to provide navigation guidance while performing a minimally invasive procedure in accordance with some aspects of the present disclosure. The method 200 is illustrated as a set of operations or processes 202-234. The processes 202-234 may be performed in the same or in a different order than the order shown in FIG. 2. One or more of the illustrated processes may be omitted in some examples of the method 200. Additionally, one or more processes that are not expressly illustrated in the flowchart may be included before, after, in between, or as part of the illustrated processes. In some examples, one or more of the processes of the flowchart may be implemented, at least in part, by a control system executing code stored on non-transitory, tangible, machine-readable media that when run by one or more processors (e.g., the processors of a control system) may cause the one or more processors to perform one or more of the processes.

At a process 202, preoperative image data is received at a control system (e.g., the control system 912 of FIG. 9). For example, a CT scan of the patient's anatomy may be performed with a conventional fan beam CT scanner, and the CT image data may be received by a control system of a robot-assisted medical system. Alternatively, preoperative image data may be received from other types of imaging systems including magnetic resonance imaging systems, fluoroscopy systems, or any other suitable method for obtaining dimensions of anatomic structures. At a process 204, a three-dimensional (3D) model of the anatomic structures (e.g., the anatomic model 106 of FIG. 1) may be constructed from the preoperative image data received by the control system. At a process 206, a target may be identified in the 3D model or in the preoperative image data from which it was constructed. For example, the target 108 of FIG. 1 may be identified in the anatomic model 106 as a region of interest for investigation or treatment. In some examples, the target may be automatically identified by the control system. The identification of the target may be confirmed by a user. For example, the control system may receive a user input confirming the identification of the target. Additionally or alternatively, the target may be visually identified by the user and manually selected or indicated in the 3D model, for example, via the display system 100 and/or via a GUI. For example, the control system may receive a user input selecting and/or indicating the target via a GUI. At a process 208, a route through anatomical passageways formed in the anatomic structures is generated. The route may be generated automatically by the control system, or the control system may generate the route based on received user inputs. The route may indicate a path along which a medical instrument (e.g., the elongate device 104 of FIG. 1) may be navigated into close proximity with the target (e.g., the target 108 of FIG. 1). The elongate device 104 may be in close proximity with the target 108 when the elongate device 104 is within a threshold distance from the target 108. In some examples, the route may be stored in the control system and incorporated into the images displayed on display system 100. For example, the route may be displayed in the virtual navigation image 102. The route may be overlaid on the anatomic model 106 in the virtual navigation image 102.

To provide accurate navigation through the anatomical passageways, a reference frame 150 of the preoperative image data (and subsequently constructed 3D model) may be registered to a reference frame of the elongate device 104 at a process 210. For example, a shape sensor (e.g., a fiber optic shape sensor or one or more position sensors) disposed along a length of the elongate device 104 may be used to provide real-time shape data (e.g., information regarding a shape of the elongate device 104 and/or a position of one or more points along the length of the elongate device 104). This shape data may be utilized to register the elongate device 104 to the 3D model constructed from the preoperative image data and to track a location of the elongate device 104 during use. Upon successful registration, a process 212 may include providing navigation guidance as the elongate device 104 is navigated through the anatomical passageways to a deployment location in proximity to the target. Navigation may be performed manually by a user with provided navigation guidance, automatically by a control system, or via a combination of both.

With the elongate device 104 positioned at or near the deployment location within the anatomy of the patient (e.g., in close proximity to the target 108), an intraoperative imaging scan may be performed. The elongate device 104 may be in close proximity with the target 108 when the elongate device 104 is within a threshold distance from the target 108. At a process 214, intraoperative image data may be received at the control system from an intraoperative imaging system. In some examples, the intraoperative imaging system may be a cone beam CT (“CBCT”) scanner than generates intraoperative CT scan image data, although any suitable imaging technique may be used without departing from the examples of the present disclosure. As compared to other imaging techniques such as conventional CT or fluoroscopy, CBCT imaging may provide a more rapid scan of a region of the patients anatomy to reduce delay of the procedure and may also have more portable and compact hardware.

The intraoperative image data may be received at the control system or other processing platform associated with the elongate device. Communication of the intraoperative image data may originate from an application programming interface of the intraoperative imaging system. As an example, the Cios Spin® imaging system marketed by Siemens® Medical Solutions USA, Inc., utilizes a protocol called NaviLink 3D™ which provides a digital interface to connect the imaging system with navigation systems and transfer datasets thereto. In some examples, the shape data associated with the elongate device may be transferred to the intraoperative imaging system, or both the shape data and the intraoperative image data may be transferred to a common platform for processing. In this regard, registration of the shape data of the elongate device to the intraoperative image data may be performed by the control system, by the intraoperative imaging system, or by another platform in operable communication with the intraoperative imaging system and/or the control system. Typically, the communication of the intraoperative image data to or from the control system will use a Digital Imaging and Communications in Medicine (“DICOM”) standard. Additionally or alternatively, the intraoperative image data may also be received in a maximum intensity projection (“MIP”) or pseudo-CT streaming format. In some examples, receiving the intraoperative image data may include receiving one or more timestamps associated with the intraoperative image data. A first timestamp may indicate the start time of the intraoperative imaging scan, and a second timestamp may indicate a stop time of the intraoperative imaging scan. In some examples, a timestamp may be associated with each instance of intraoperative image data. In order to ensure accurate correlation, a clock of the control system associated with the elongate device may be synchronized with a clock associated with the intraoperative imaging system. Each instance of shape data may be associated with a timestamp. In some examples, each timestamped instance of intraoperative image data may be paired with a correspondingly timestamped instance of shape data.

In order to register the intraoperative imaging data to the elongate device, while the intraoperative imaging data is captured, at a process 216, shape data from the elongate device may be received by the control system, for example, during the intraoperative imaging procedure. The shape data may be captured for only a brief period of time or may be captured during the entire image capture period of the intraoperative imaging scan. The shape data may be captured for any other period of time during the image capture period of the intraoperative imaging scan. A variety of synchronizing techniques may be used to ensure that only shape data corresponding to the image capture period is used for registration, even though shape data outside the image capture period may also be received.

At a process 218, the intraoperative image data, or a portion thereof, may be segmented. For example, discrete units of the intraoperative image data (e.g., pixels and/or voxels) may be analyzed to assign an intensity value to each unit. Discrete units having the same or similar intensity values may be aggregated to form components. Morphological operations may be utilized to interconnect non-contiguous components having similar intensity values. In some examples, computer software, alone or in combination with manual input, is used to convert the intraoperative image data into a segmented two-dimensional or three-dimensional composite representation or model of a partial or an entire anatomic organ or anatomic region. The model may describe the various locations and shapes of the anatomical passageways and their connectivity. More specifically, during the segmentation process the pixels and/or voxels may be partitioned into segments or elements or be tagged to indicate that they share certain characteristics or computed properties, such as color, density, intensity, and texture. In some examples, segmenting the intraoperative image data may comprise selecting components to associate with certain objects. For example, segmenting the intraoperative image data associated with the elongate device may include selecting imaging units or components based upon one or more factors including proximity to the target, the shape data, an approximate registration of the elongate device to the patient, and an expected elongate device intensity value. An expected elongate device intensity value may include a range of values associated with materials from which the elongate device is composed. In some examples, an algorithm (e.g., a Gaussian Mixture Model) may be used to establish the expected elongate device intensity. In some examples, segmenting the intraoperative image data may further comprise utilizing processes established by the control system using deep learning techniques. The intraoperative image data corresponding to the elongate device may be segmented or filtered out of the intraoperative image data. A model of the shape of the elongate device may be generated based on the segmented intraoperative image data corresponding to the elongate device. For example, the elongate device may be identified as an elongate device in the intraoperative image data by the segmentation. Additionally or alternatively, the elongate device may be identified as an elongate device in the intraoperative image data by filtering the intraoperative image data by CT number or Hounsfield value associated with the elongate device. This data associated with the elongate device may be isolated from other portions of the intraoperative image data that are associated with the patient or with specific tissue types. A three-dimensional mesh model may be formed around the isolated data and/or a centerline may be determined that represents a centerline of the elongate device. The segmented intraoperative image data for the elongate device may be expressed in the intraoperative image reference frame.

In some examples, information about the elongate device may be used to seed the segmentation process. For example, an elongate device (e.g., a steerable catheter) may include a metal spine embedded in a non-metal sheath. High contrast information in the intraoperative image data associated with the spine may be identified first, and a region around the spine may be searched for the non-metal sheath in pixels or voxels having less contrast. In some examples, a high-contrast fiducial marker may be inserted through a working channel of the elongate device during intraoperative imaging to improve segmentation of the elongate device.

At a process 220, the target (e.g., the target 108 of FIG. 1) may be identified in the intraoperative image data. In some examples, when the elongate device has already been segmented or identified, identifying the target may comprise establishing a region of interest (e.g., a subregion) in the intraoperative image data within a predetermined range of the elongate device. The region of interest, rather than all of the intraoperative image data, may be analyzed to segment the target from the region of interest. In some examples, the search field in which to locate the target may be reduced based upon an assumption that the elongate device was previously navigated into close proximity with the target. In some examples, identifying the target may include receiving an indication or selection from a user at a user interface, such as a GUI. For example, the control system may receive a user input manually selecting portions of the intraoperative image data associated with one or more components on the display system to associate with the target. Manual identification of the target may be necessary when automatic identification of the target has produced unsatisfactory results. In some examples, the control system may instruct a user to manually identify the target in the intraoperative image data. In some examples, identifying the target in the intraoperative image data may result in registering the preoperative image data (or the 3D model 106) to the intraoperative image data based on a preoperative location of the target and an intraoperative location of the target.

At a process 222, the intraoperative image data may be registered to the shape data of the elongate device by comparing the shape data received from the elongate device to the portion of the intraoperative image data corresponding to the elongate device. The shape data from the elongate device may be expressed in the elongate device reference frame 350 and/or the surgical reference frame 250. This registration may rotate, translate, or otherwise manipulate by rigid or non-rigid transforms points associated with the segmented shape of the elongate device and points associated with the shape data of the elongate device. In some examples, this registration may be performed using an iterative closest point algorithm or another point cloud registration technique. Optionally, data points may be weighted based upon segmentation confidence or quality to assign more influence to data points that the control system determines are more likely to be accurate. Additionally or alternatively, registering the intraoperative image data to the shape data of the elongate device may be performed using coherent point drift or an uncertainty metric (e.g., RMS error). In some examples, the segmented shape of the elongate device is registered to the shape data and the associated transform, such as a vector applied to each of the points in the segmented shape to align with the shape data in the shape sensor reference frame, may be applied to the entire three-dimensional image and/or to subsequently obtained three-dimensional images during the medical procedure. The transform may be a six degrees-of-freedom (6DOF) transform, such that the shape data may be translated or rotated in any or all of X, Y, and Z and pitch, roll, and yaw. Discussion of processes for registering an elongate device to image data may be found, for example, in Intl. Pat. App. Pub. No. WO 2021/092116, filed Nov. 5, 2020, entitled “Systems and Methods for Registering an Instrument to an Image Using Change in Instrument Position Data” and Intl. Pat. App. Pub. No. WO 2021/092124, filed Nov. 5, 2020, entitled “Systems and Methods for Registering an Instrument to an Image Using Point Cloud Data,” both of which are incorporated by reference herein in their entireties.

When the intraoperative image reference frame is registered to the elongate device reference frame, the images displayed on the display system 100 (e.g., the virtual navigation image 102) may allow an operator to: more accurately navigate the elongate device through the patient anatomy, visualize a target lesion relative to the elongate device, observe a view from the perspective of a distal end of the elongate device, and/or improve the efficiency and efficacy of targeted medical procedures.

In some examples, the intraoperative image data may be registered with preoperative image data obtained by the same or a different imaging system. Thus, by registering the shape data to the intraoperative image data, the registration of the shape data to the preoperative image data may also be determined. In some examples, an anatomic image generated from the intraoperative image data and/or the preoperative image data may be displayed with the image of the elongate device derived from the shape data received from the elongate device. For example, a model of the elongate device generated from the instrument shape data may be superimposed on the image of the patient anatomy generated from the preoperative or intraoperative image data.

At a process 224, the intraoperative location of the target may be mapped to the elongate device reference frame based on the registration performed in process 222. In some examples, the intraoperative location of the target may be compared to the preoperative location of the target. Additionally or alternatively, the preoperative geometry (e.g., surface boundaries) of the target may be compared to the intraoperative geometry of the target. If there is a discrepancy, the target location or geometry may be updated within the model 106 so that the preoperative location and/or geometry of the target is modified to match the intraoperative location and/or geometry of the target at a process 226. The updated location of the target may be displayed in the preoperative image reference frame, such that the updated location target is displayed in the 3D model 106 on the display system 100.

At a process 228, the GUI may display a graphical indicator overlaid on the intraoperative image data. In some examples, the graphical indicator illustrates a target deployment location for a distal tip of a medical instrument that is deployed through the elongate device. At a process 230, a treatment zone may be overlaid on the intraoperative image data in the GUI. The treatment zone may indicate the area where tissue and other anatomic structures may be affected by a treatment. For example, the treatment zone may indicate an ablation zone, which may indicate the area where tissue and other anatomic structures may be affected by an ablation procedure. Additionally or alternatively, the treatment zone may indicate a radiation zone, which may indicate the area where tissue and other anatomic structures may be affected by a radiation procedure. Additionally or alternatively, the treatment zone may indicate a fiducial zone, which may indicate the area where one or more fiducials may be placed in, on, or around tissue and other anatomic structures. The treatment zone may indicate the area where tissue and other anatomic structures may be affected by any other treatment or procedure. The treatment zone may indicate an area of focus within or surrounding an anatomic structure, such as a target anatomic structure for a biopsy procedure. In some examples, more than one treatment zone may be overlaid on the intraoperative image data in the GUI.

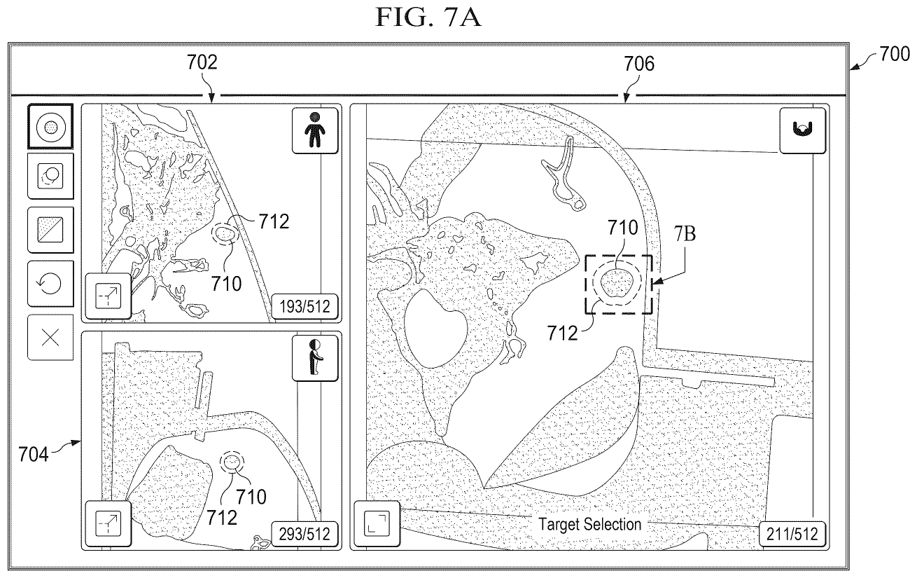

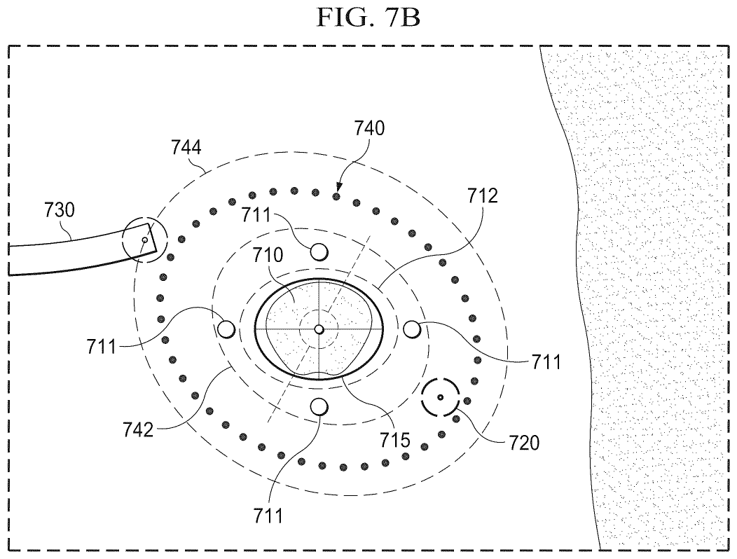

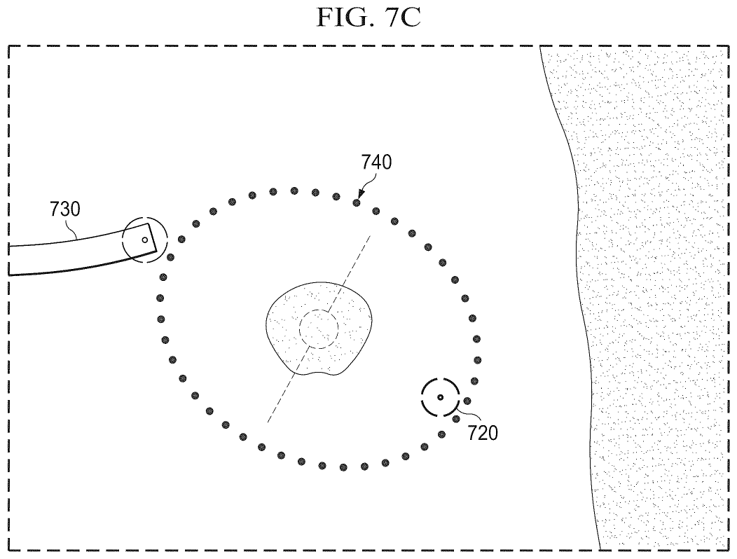

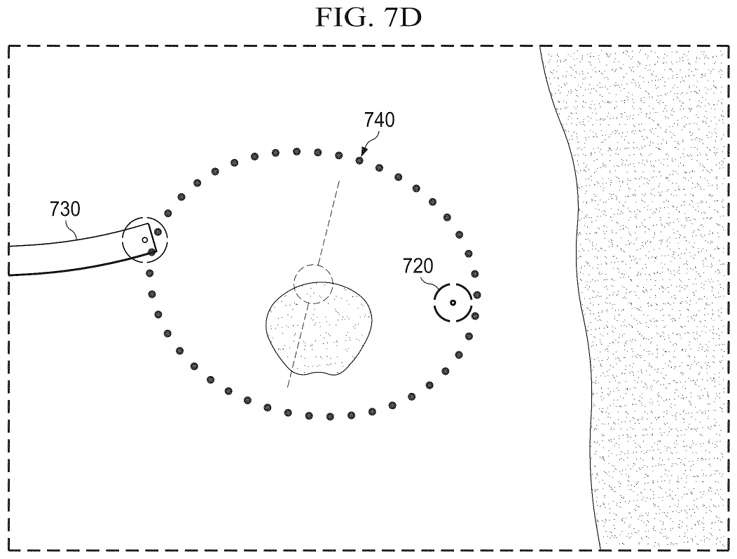

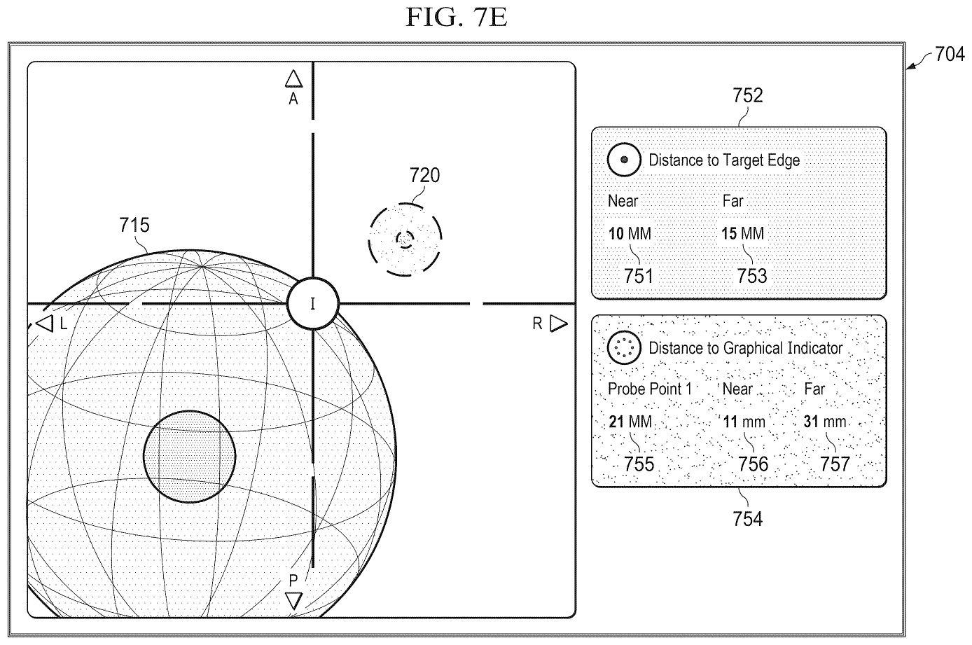

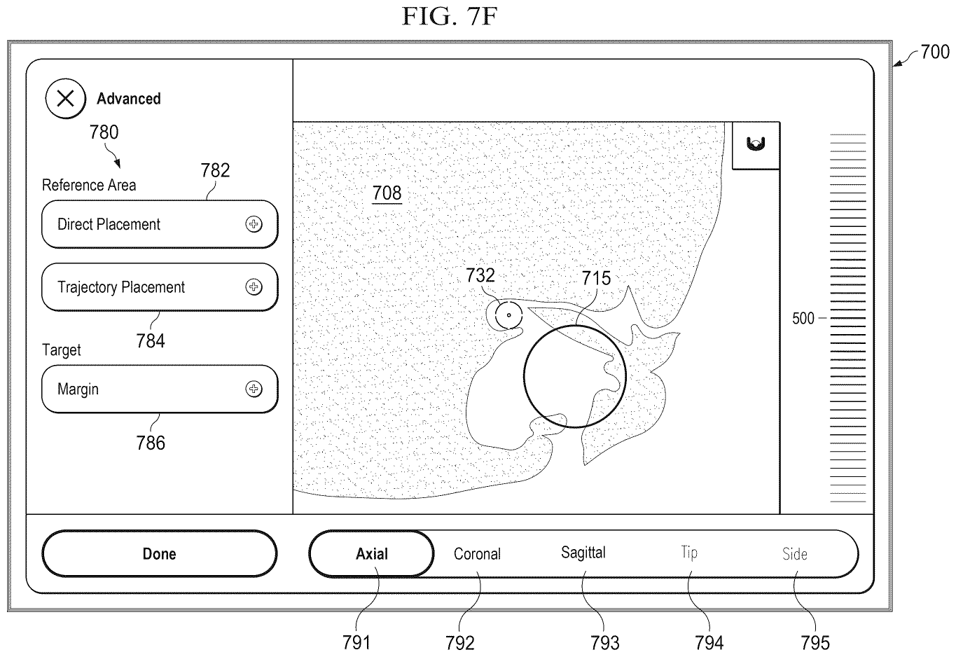

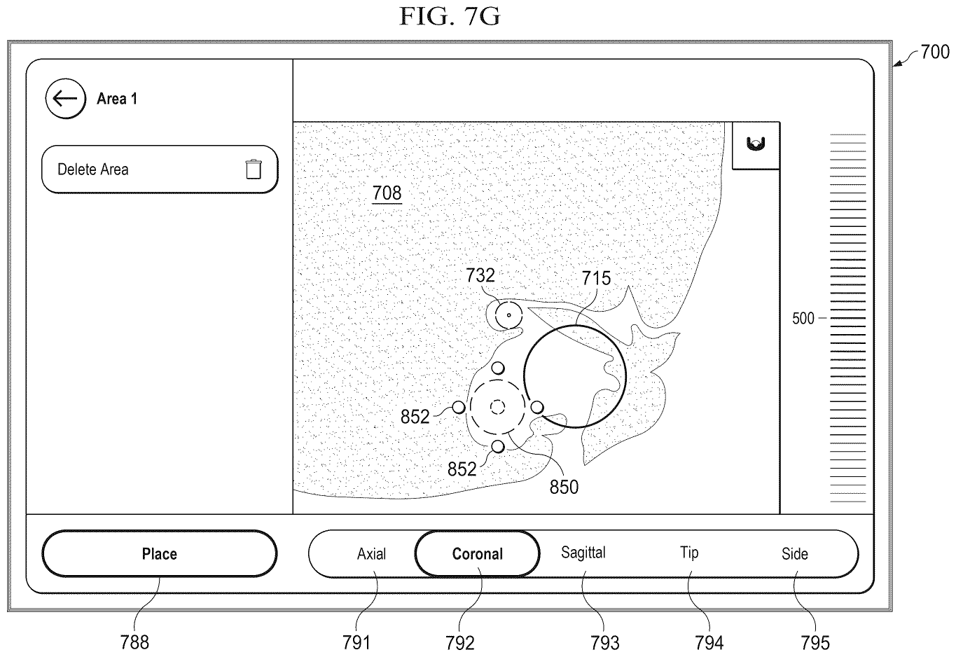

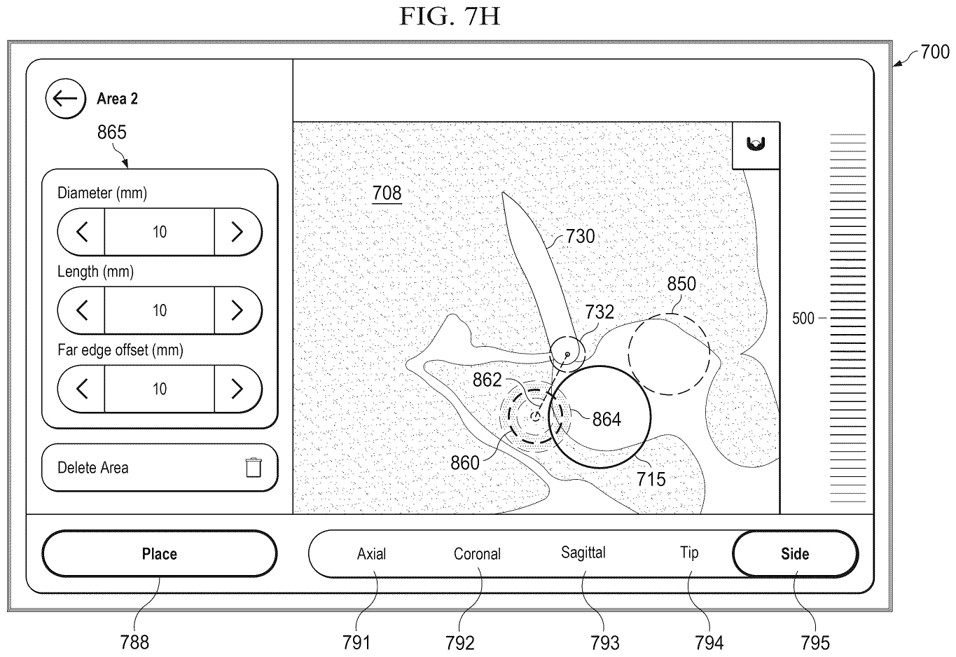

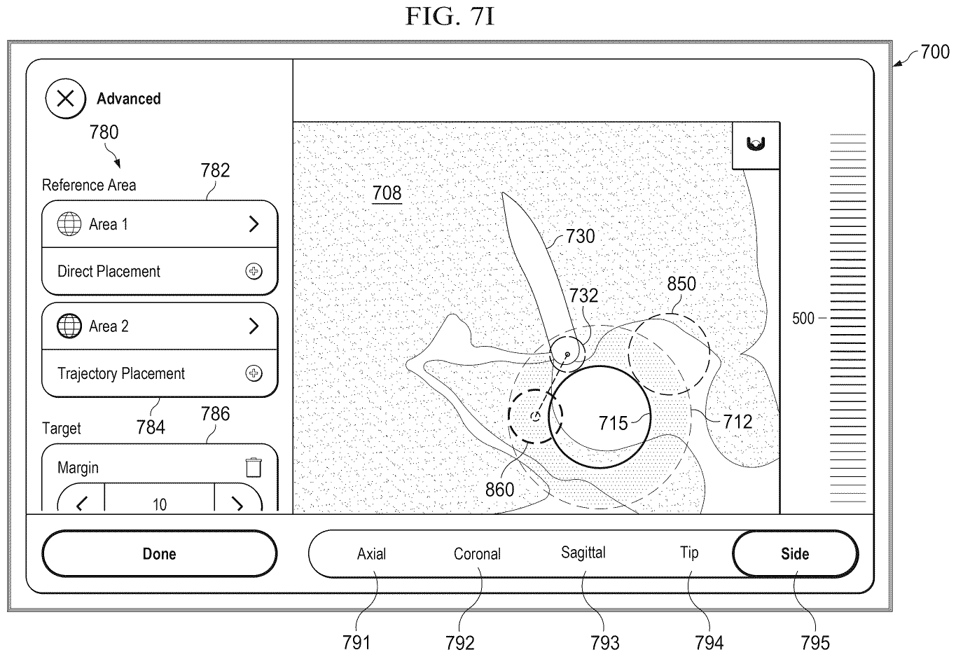

At a process 232, intraoperative image data may be received at the control system from an intraoperative imaging system while the medical instrument is deployed (e.g., extended out from a distal tip of the elongate device). In some examples, the intraoperative imaging data may be received from an intraoperative external imaging system, such as a CBCT system, although any suitable imaging technique may be used without departing from the examples of the present disclosure. At a process 234, the control system may receive confirmation that the elongate device is retracted. Further details regarding processes 228-234 will be described below with reference to FIGS. 7A-7E.

In some examples, portions of the intraoperative image data corresponding to one or more anatomical passageways may be segmented using similar segmentation techniques as those discussed above. The segmentation of the anatomical passageways may result in revised surface boundaries, diameters, locations, etc. of one or more anatomical passageways. After registering the intraoperative image data to the shape data, the revised geometry and/or location of at least one of the anatomical passageways may be updated in the 3D model displayed on the graphical user interface from a preoperative configuration to an intraoperative configuration based on the intraoperative image data. The updated configuration of one or more anatomical passageways may result in an updated navigation path of the elongate device. For example, intraoperative imaging may indicate that an anatomical passageway previously believed to be too narrow for safe navigation of the elongate device may, in fact, be larger in diameter than was indicated by the preoperative imaging. By updating the diameter of the anatomical passageway in the model 106, the control system may determine that the revised anatomical passageway provides a more direct route to the target. The process of updating anatomical structures is not limited to anatomical passageways (e.g., airways). For example, intraoperative image data may be utilized to revise anatomy borders of tissue and/or organs in the model 106 (e.g., pleura, lung fissures, vasculature, etc.).

Segmentation and updating of the target, anatomical passageways, and/or other anatomical structures may be performed automatically by the control system independent of user input. Additionally or alternatively, these processes may be initiated by a received user input to identify one or more locations in the intraoperative image data corresponding to the respective feature(s).

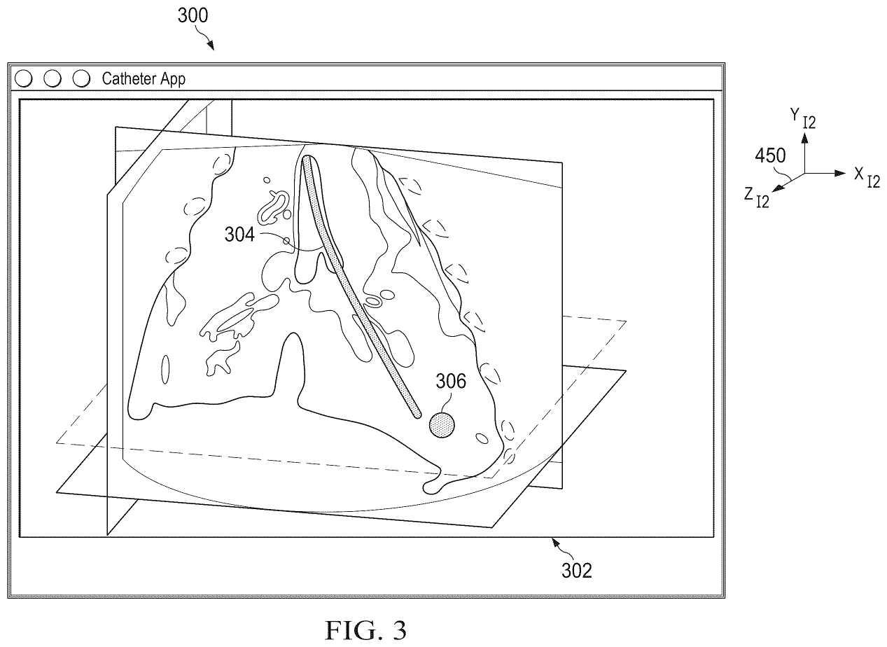

In some examples, the intraoperative image data received at process 214 of FIG. 2 may be displayed on a graphical user interface (GUI) 300 of a display system as shown in FIG. 3. The segmented elongate device may be displayed in conjunction with the intraoperative image data on the display system. Additionally or alternatively, the segmented target may be displayed in conjunction with the intraoperative image data on the display system. FIG. 3 illustrates a viewing mode 302 providing a two-dimensional and/or three-dimensional view of the intraoperative image data in which the elongate device 304 and the intraoperative target 306 (which may be the same as the preoperative target 108) have been segmented. The intraoperative image data is displayed in an intraoperative image reference frame (X_{I2}, Y_{I2}, Z_{I2}) 450. Pixels or voxels may be displayed with assigned intensity values which provide an initial visual demarcation between distinct structures. The viewing mode 302 may display the segmented elongate device centerline and/or boundary registered to the 3D model 106. When segmentation is unsuccessful, the GUI 300 may allow a user to manually identify the elongate device and/or the target. In some examples, the elongate device 304 and/or the target 306 may be displayed in a different color or may be otherwise visually distinguished from surrounding anatomical structures.

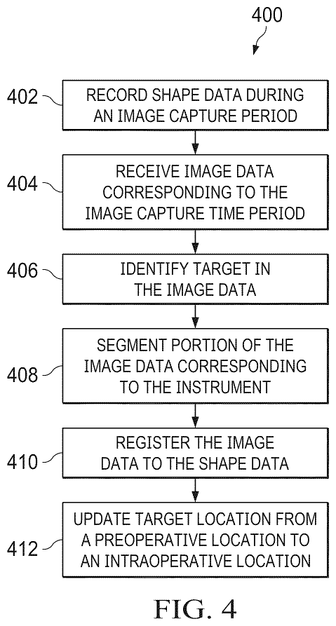

As discussed above with reference to process 222 of FIG. 2, the intraoperative image reference frame 450 may be registered to the elongate device reference frame 350. As discussed above with reference to process 226 in FIG. 2, a target location may be updated from a location based on preoperative image data to a location based on intraoperative image data. FIG. 4 illustrates a method 400 of registering intraoperative image data to shape data from an elongate device (e.g., the elongate device 104) to update a location of a target (e.g., the target 108) in a model (e.g., the model 106). At a process 402, elongate device shape data may be recorded during an image capture period of an imaging system. The imaging system may be a cone beam CT system or any other imaging system configured for capturing intraoperative images of an instrument and patient anatomy. At a process 404, image data corresponding to the image capture period may be received. The image data may include one or more of the patient anatomy, the target, and the elongate device. At a process 406, the target is identified in the image data. For example, the target may be segmented by the control system or other processing platform. Additionally or alternatively, the control system may receive a user input identifying the target. At a process 408, a portion of the image data corresponding to the elongate device may be segmented. Using the segmented image data and the shape data recorded in process 402, the image data may be registered to the shape data based on the shape of the elongate device during the image capture period at a process 410. At a process 412, using the registered image data, the location of the target may be updated from a preoperative location based on preoperative imaging to an intraoperative location based on the intraoperative imaging. The updated target location may improve navigation of the elongate device to the target.

In some examples, an additional or alternative process may be used to update a location of an anatomical passageway in the preoperative image reference frame 150. For example, one or more portions of the image data corresponding to anatomical passageways may be segmented. Using the segmented image data and the shape data recorded in process 402, the image data may be registered to the shape data based on the shape of the elongate device during the image capture period. Using the registered image data, the location of one or more anatomical passageways may be updated from a preoperative location based on preoperative imaging to an intraoperative location based on the intraoperative imaging. Updating of the one or more anatomical passageways may provide a more accurate path from a current location of the elongate device to the target. It should be appreciated that method 400 is optional.

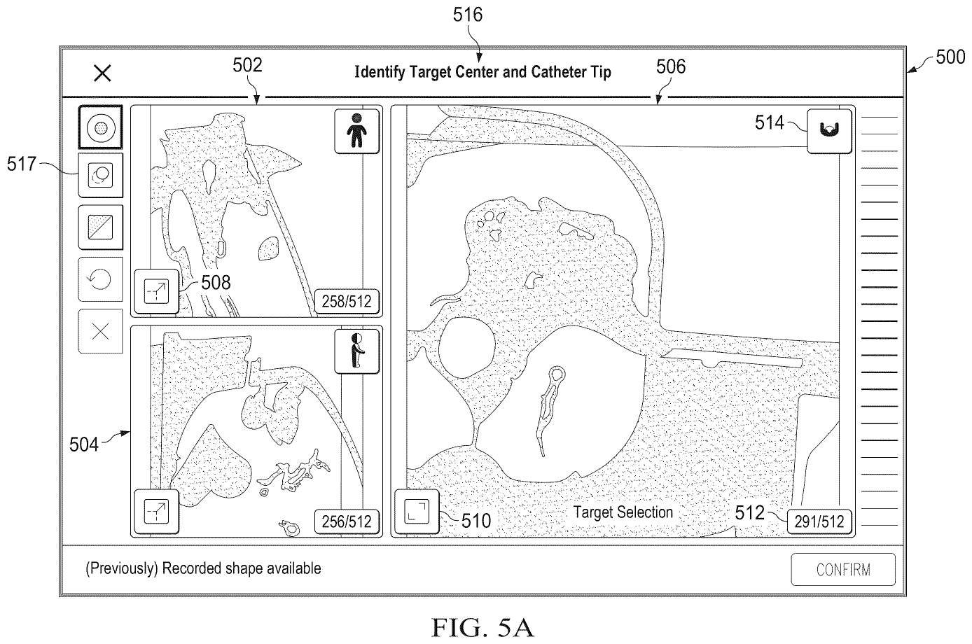

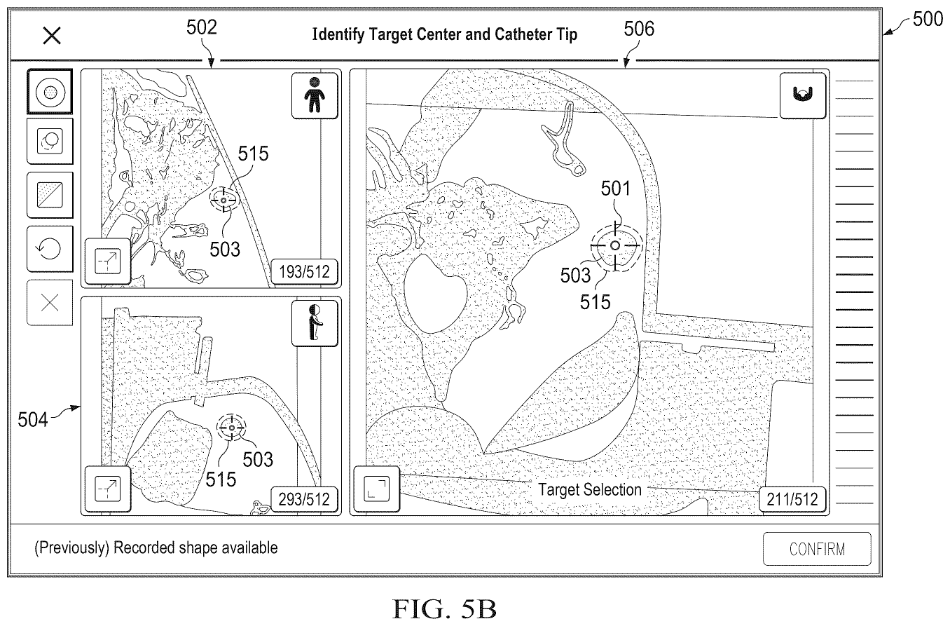

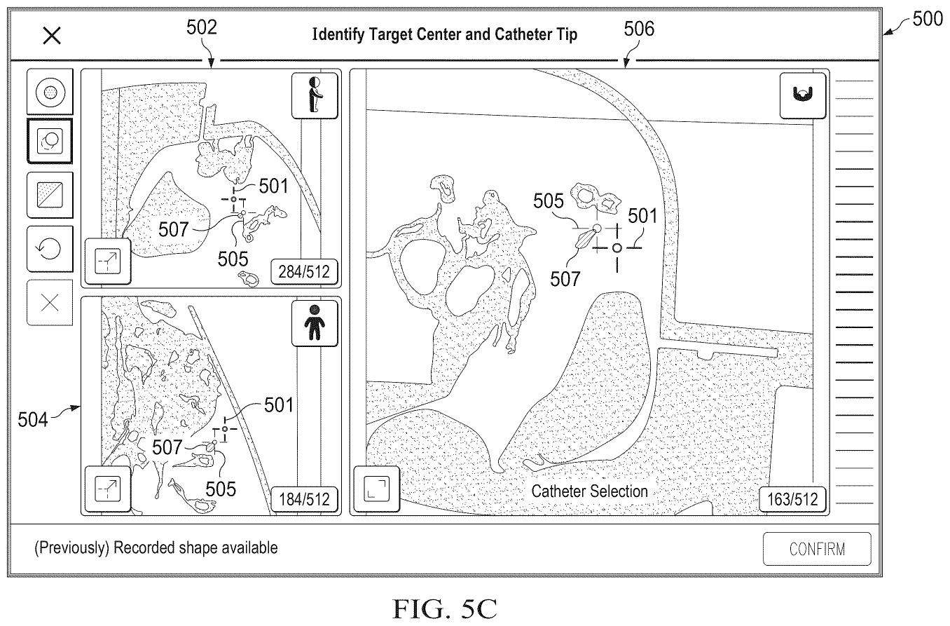

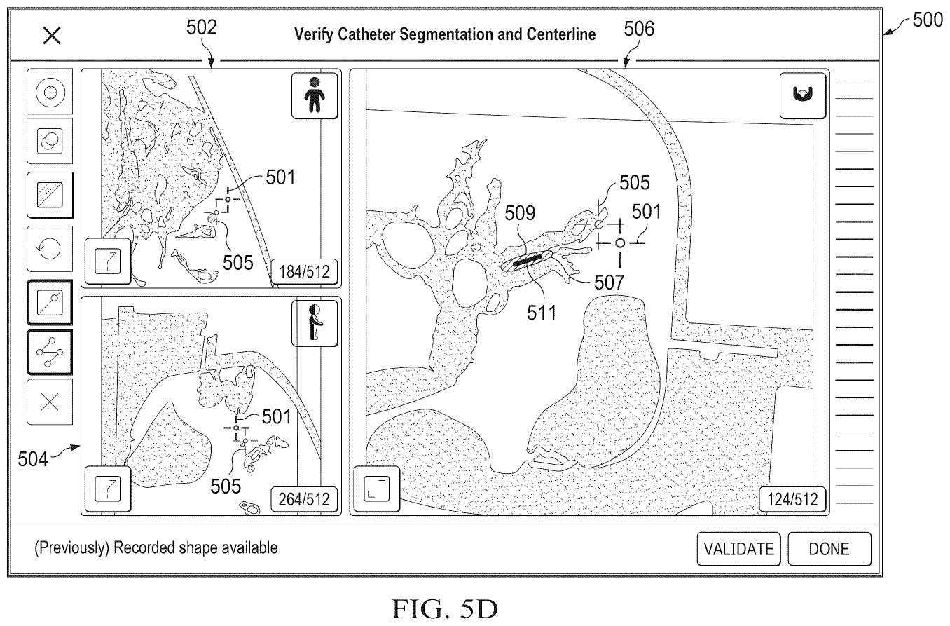

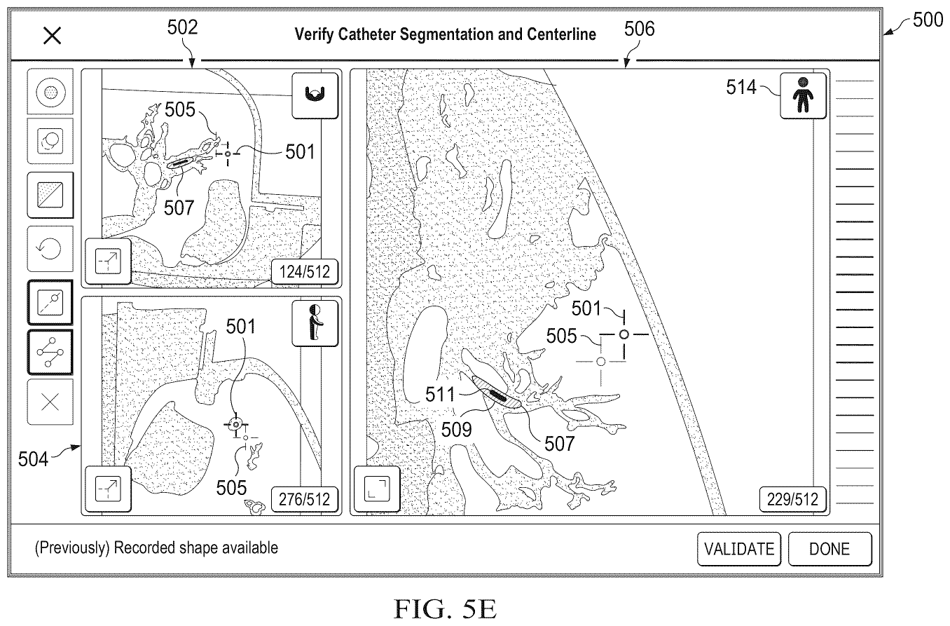

An example of a GUI for performing various processes discussed in above in relation to FIG. 2 is illustrated in FIGS. 5A-5E. A GUI 500 may include a plurality of view windows for displaying visual information to a user. For example, FIG. 5A illustrates the GUI 500 including view windows 502, 504, and 506 illustrating cross sections of the intraoperative image data taken along coronal, transverse, and sagittal planes, respectively. Although three view windows are illustrated, more or less view windows may be displayed at any given time.

In FIG. 5A, a primary view window 506 is larger than two secondary view windows 502 and 504. The control system may receive a user input selecting the visual information to be displayed in the primary view window 506. For example, a user input selecting a maximize icon 508 on one of the secondary view windows 502, 504 may be received. In some examples, the control system may receive a user input to enlarge the information shown in the primary view window 506 to a full-screen view based on receiving a user input selecting the full-screen button 510. A view indicator 514 may be overlaid on each view window 502, 504, 506 to indicate the plane or orientation to which the currently displayed images correspond.

Each view window may allow a user to scroll through a series of consecutive intraoperative images taken along parallel planes, for example, by hovering a cursor over a given view window and rotating a scroll wheel or other input device. An image indicator 512 may indicate the current image being displayed and the total number of images available for each view. A toolbar 517 may be displayed in the GUI 500 to allow a user to adjust various properties of the displayed visual information such as zoom, contrast, etc.

FIG. 5A illustrates a prompt 516 displayed in the GUI 500 stating “Identify Target Center and Catheter Tip.” As shown in FIG. 5B, and with regard to process 220 of FIG. 2, the control system may receive a user input selecting the target. The user input may be, for example, clicking a mouse or touching a touchscreen, but may be one or more additional or alternative user inputs (e.g., an audible input, a gaze-tracked input, a text input, etc.). The user may scroll through each series of images until the target is visible prior to selecting the target. For example, in FIG. 5A, the target does not appear to be visible in the GUI 500, but the target 503 is visible in FIG. 5B. By comparing the image indicators 512 of each view window between FIGS. 5A and 5B, it can be seen that the user has scrolled through each series of images until the target 503 is visible. For example, FIG. 5A shows the view window 506 displaying image “291/512,” and FIG. 5A shows the view window 506 displaying “image 211/512.” A target icon 501 may be generated and displayed at the selected location in each of the view windows. This process may be repeated for each of the view windows 502, 504, 506. Alternatively, in some examples, the target 503 may be selected in only one view window, and the control system may automatically identify the target 503 in the other view windows. As another example, the control system may identify the target 503 independent of any user input based on known or anticipated properties of the target 503 (e.g., expected pixel or voxel intensity value).

A margin 515 (e.g., the margin 110) may be overlaid on the intraoperative image data and displayed in one or more of the view windows 502, 504, 506. The margin 515 may surround the target 503 and optionally may act as a safety margin to provide the user guidance for treating (e.g., ablating) diseased cells. The margin 515 may be sized and shaped to account for portions of the target 503 that may not be visible in the intraoperative image data. In some examples, only one slice of the intraoperative image data is displayed in the GUI 500 at a time, and portions of the target 503 might only become visible in other slices of the image data as the user scrolls through the slices. The margin 515 may alternatively or additionally be sized to account for any computational error and/or execution error that may be present when determining the size and shape of the target 503.

In some examples, the margin 515 may be uniform around the entirety of the target 503. For example, the margin 515 may provide a 5 mm buffer around the target 503. The margin 515 may provide a buffer of any other size, such as 3 mm, 8 mm, 10 mm, or any other size that may encompass portions of the target 503 that may not be visible in the particular slice of the intraoperative image data shown in the view window 506, for example. In some examples, one or more portions of the margin 515 may be non-uniform around the entirety of the target 503 and may be different lengths at different parts of the target 503. For example, one portion of the margin 515 may provide a 5 mm buffer and another portion of the margin 515 may provide a 3 mm buffer. The margin 515 may be non-uniform due to, for example, the type of target 503, the proximity of one or more critical structures (e.g., lung pleura, lung fissures, blood vessels, the heart, etc.), the proximity of anatomical structures (e.g., anatomical passageways, bones, etc.), or any other similar factor.

In some examples, the size of the margin 515 may be automatically set and/or adjusted by a control system based on the type of target 503 to be treated. For example, the control system may retrieve information from a target database that includes dimensions and other physical characteristics of different types of anatomical targets. Additionally or alternatively, an image processor of the control system may perform image analysis of the intraoperative image data to determine the size and shape of the target 503.

In some examples, the size of the margin 515 may be set and/or adjusted by a user. The size and/or shape of the margin 515 may be adjusted or altered to account for patient movement (e.g., respiratory movement or circulatory movement) and/or CT to body divergence. For example, the control system may receive a user input via a menu (not shown) of the GUI 500 that adjusts the size of the margin 515. In some examples, a table or menu (not shown) may be displayed in the GUI 500 that includes a list of spheres and/or ellipses with pre-specified sizes. The control system may receive a user input selecting one or more of the spheres and/or ellipses. The selected spheres and/or ellipses may be overlaid on the intraoperative image data and displayed in one or more of the view windows 502, 504, 506. In some examples, the control system may receive the user input via the GUI 500. Additionally or alternatively, the user input may be numerical values defining the distance the margin 515 should expand beyond the outer edges of the target 503.

With reference to FIG. 5C, the control system may receive a user input selecting a location associated with an elongate device 507 (e.g., the elongate device 104) in one or more of the view windows. As with the target 503, the user may scroll through the respective images of each view window until the elongate device 507, or a portion thereof, is visible in the view window(s). In the illustrated example, the distal tip of the elongate device 507 has been selected in each view window 502, 504, 506. An elongate device icon 505 has been generated at each selected location corresponding to the distal tip of the elongate device 507. The selected location of the distal tip of the elongate device 507 may be used to seed the segmentation process (e.g., process 220 of FIG. 2). For example, during segmentation, the control system may seek voxels having intensity values similar to the intensity value of the voxel corresponding to the distal tip of the elongate device 507. Additionally or alternatively, the control system may identify and segment the elongate device 507 from the intraoperative image data automatically. In such an example, the control system may populate the view windows of the GUI 500 with the respective elongate device icons 505. The control system may request confirmation from the user to confirm that the identified location of the distal tip of the elongate device 507 is accurate.

Following segmentation, the GUI 500 may display the segmented elongate device 507 overlaid on the intraoperative image data as shown in FIG. 5D. One or more of the view windows 502, 504, 506 may display an elongate device boundary 509 and/or an elongate device centerline 511. The user may scroll through the series of images and verify that the segmentation process appears to have rendered satisfactory results. For example, FIG. 5E illustrates a different set of images in each view window 502, 504, 506 as compared to FIG. 5D, each illustrating the segmented elongate device 507, target icons 501, and elongate device icons 505.

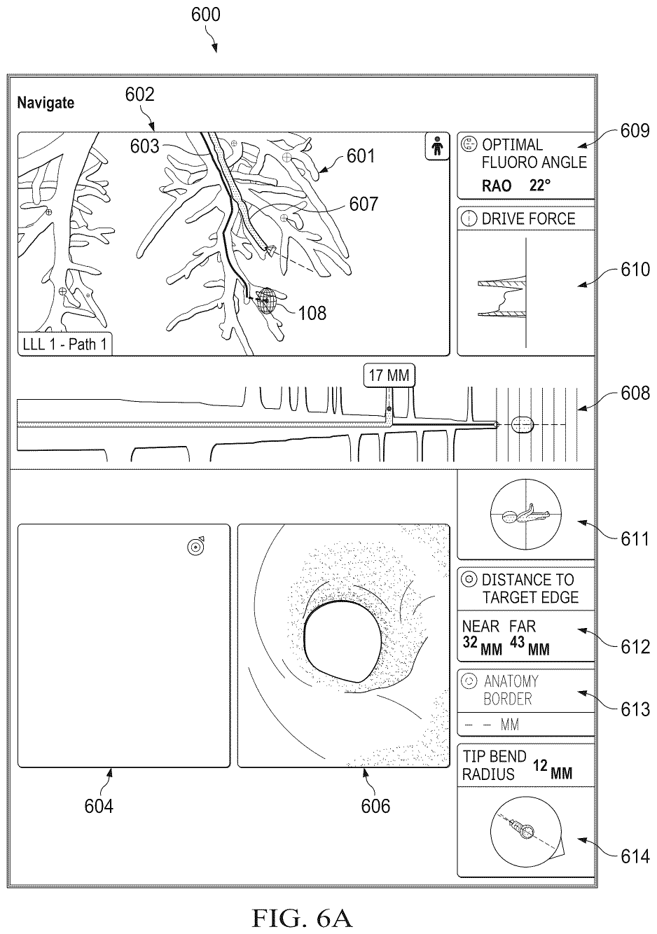

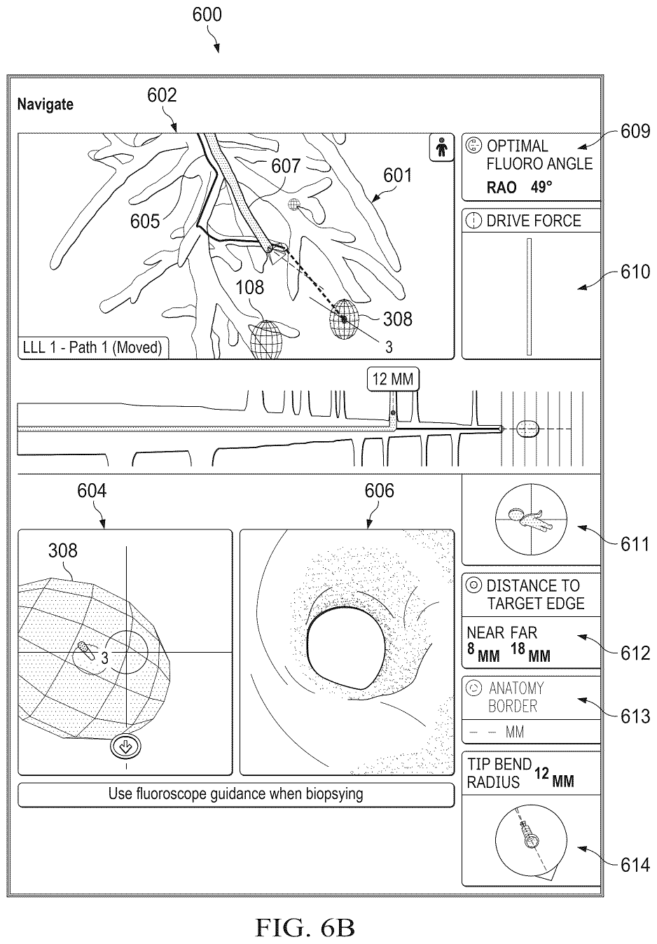

A GUI 600 before and after the updating process 226 of FIG. 2 is shown in FIGS. 6A and 6B. As shown in FIG. 6A, the GUI 600, which may be same as or similar to the GUI 500, includes a virtual navigation image 602. The virtual navigation image 602 may display a 3D model 601. In some examples, an elongate device 607 (e.g., the elongate device 507) and/or the target 108 may be overlaid on the 3D model 601. A navigation path 603, as determined in process 208, may also be overlaid on the 3D model 601.

The GUI 600 may also include a virtual camera view 604 and/or a physical camera view 606. The physical camera view 606 displays video from a camera disposed within the anatomy of the patient. For example, the elongate device 607 may include an endoscope, and the physical camera view 606 may display a video feed from an endoscope camera. The virtual camera view 604 may display a computer-generated image of the target 108 from a perspective of the distal tip of the elongate device 607. As can be seen in the virtual navigation image 602, the distal tip of the elongate device 607 is not directed at the location of the target 108. Accordingly, there is no computer-generated image of the target 108 displayed in the virtual camera view 604 in the example shown in FIG. 6A.

Additionally or alternatively, the GUI 600 may include a navigation overview 608 illustrating widths and branching relationships of various anatomical passageways along the length of the navigation path 603, as well as the progress of the elongate device 607 along the navigation path 603. Any one or more of various other graphics, icons, indicators, or windows may also be included in the GUI 600, such as an optimal fluoroscopic angle graphic 609, a drive force graphic 610, an orientation indicator 611, a target distance indicator 612, an anatomy border distance indicator 613, a tip bend radius graphic 614, or any combination thereof. The optimal fluoroscopic angle graphic 609 displays a suggested positioning angle for a fluoroscopic imaging system with respect to the patient to optimize imaging of the target 108 (e.g., to minimize obstructions). The optimal fluoroscopic angle graphic 609 may additionally or alternatively illustrate the optimal imaging angle for any external imaging system, such a fluoroscopic imaging system, a cone beam CT imaging system, etc. The drive force graphic 610 displays the forces applied (e.g., as measured at an actuator or as measured or estimated at the distal tip of the elongate device 607) to navigate the elongate device 607 to its current location. The orientation indicator 611 displays the current orientation of the distal tip of the elongate device 607 corresponding to the views provided by virtual camera view 604 and/or physical camera view 606. The target distance indicator 612 illustrates a current distance between the distal tip of the elongate device 607 and the target 308. In the illustrated example, the target distance indicator 612 provides a distance from the distal tip of the elongate device 607 to the nearest point of the target 108 and a distance from the distal tip of the elongate device 607 to the furthest point of the target 108. In some examples, a single distance may be provided such as a distance from the distal tip of the elongate device 607 to a central point of the target 108 or to a specific point of interest within the target 108. The anatomy border distance indicator 613 indicates a current distance between the distal tip of the elongate device 607 and the closest anatomy element, such as the pleura of the lungs, a fissure of the lungs, blood vessels, etc., in the direction of the tip of the elongate device 607. The tip bend radius graphic 614 indicates a current bend radius of the distal tip of the elongate device 607 which may be used during navigation to prevent over-bending of the elongate device 607. Over-bending of the elongate device 607 may cause damage to the elongate device 607 or to patient tissue.

FIG. 6B illustrates the GUI 600 after the location of the target is updated from the location of target 108 to a location of target 308. In some examples, the control system may determine an updated navigation path 605 to the updated location of the target 308. Additionally, the location of at least a portion (e.g., the distal tip) of the elongate device 607 within the anatomical passageways of the model 601 may be updated. In some examples, the updated location of the target 308 may be within the field of view of the virtual camera in the virtual camera view 604 as shown in FIG. 6B.

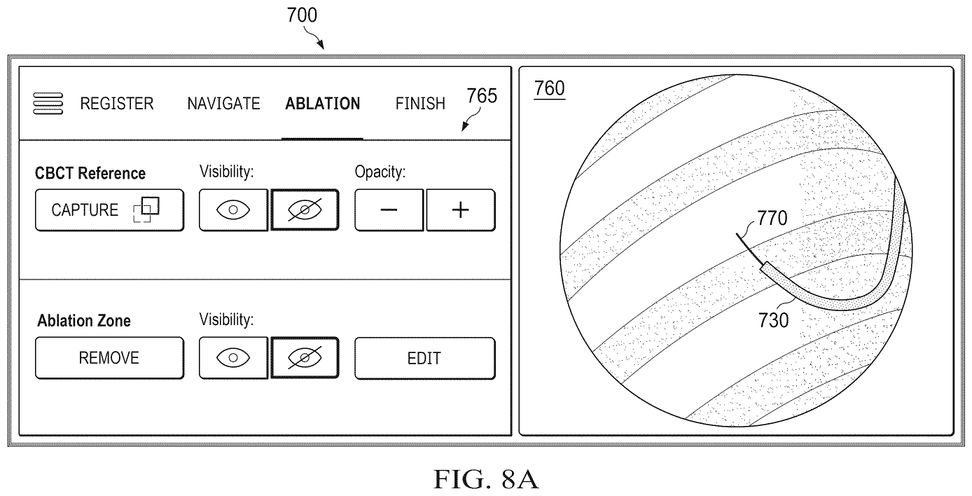

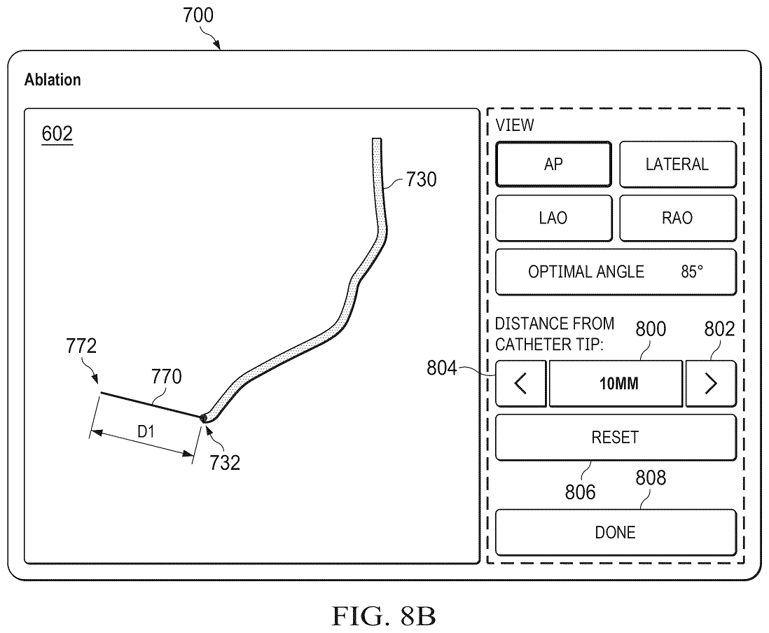

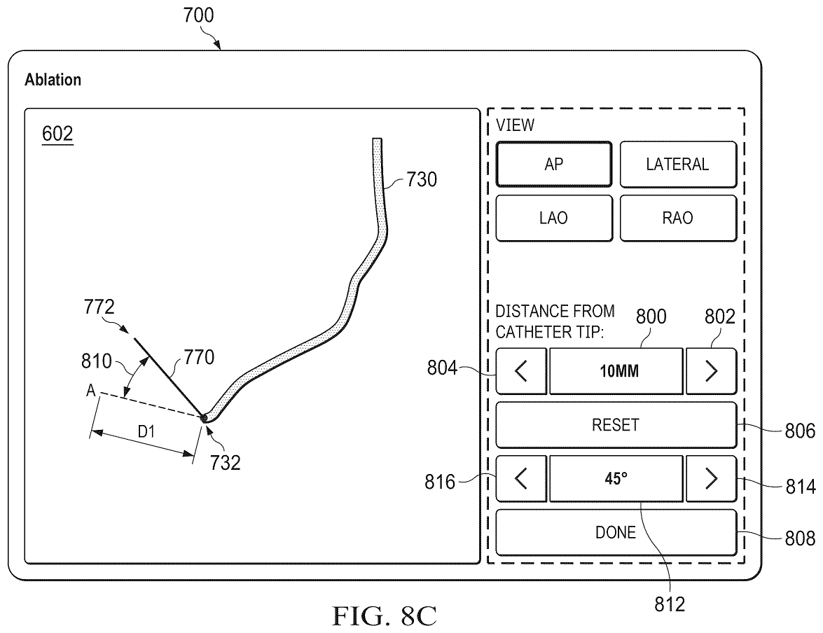

In some examples, the elongate device 607 may include a working channel through which a medical instrument (e.g., the medical instrument 870 of FIG. 8A, which may be an ablation probe, a biopsy needle, an ultrasound transducer, etc.) may be inserted. Segmentation of the elongate device 607 may include segmentation of the medical instrument as well. The segmentation process discussed above with respect to process 218 of FIG. 2 in the context of segmenting the elongate device may similarly apply to segmenting the medical instrument. In this regard, the medical instrument may be separately identifiable from the elongate device 607 in the GUI 600. When the medical instrument is extended or protruding from the elongate device 607 (e.g., extending or protruding from the distal tip of the elongate device 607) during an intraoperative imaging procedure, the control system may analyze the segmented target 308 and segmented medical instrument to determine whether the medical instrument is positioned at a desired deployment location. Additionally or alternatively, the control system may receive a user input indicating whether the medical instrument is positioned at the desired deployment location.

In some examples, the displaying process 228 of FIG. 2 may be performed after the updating process 226 of FIG. 2. Alternatively, the displaying process 228 of FIG. 2 may be performed after identifying the target 503 in the intraoperative image data as discussed above with respect to process 220 of FIG. 2. FIG. 7A illustrates a GUI 700 for performing various processes discussed above in relation to FIG. 2. The GUI 700 may be the same as or similar to the GUI 500. The GUI 700 includes view windows 702, 704, and 706. Although three view windows are illustrated, more or fewer view windows may be displayed at any given time.

In some examples, the view window 706 illustrates an oblique view of the patient anatomy. The oblique view may allow for the user to more easily view the target 710, an elongate device 730, and a treatment zone 740 (e.g., an ablation zone) when scrolling through the slices of the CBCT image data, which will be discussed in further detail below.

As discussed above with respect to the GUI 500, the intraoperative image data displayed in the GUI 700 may include an image of an anatomical target 710 (e.g., the target 108). A margin 712 (e.g., the margin 515) may be overlaid on the intraoperative image data and displayed in one or more of the view windows 702, 704, 706. FIG. 7B illustrates a close-up view of an intraoperative image data that includes the target 710 and the surrounding area of the patient anatomy in close proximity to the target 710. Some or all of the features discussed below with respect to FIG. 7B may be displayed in the GUI 700 shown in FIG. 7A.

In some examples, the size and/or orientation of the target 710 may be adjusted based on the image of the target 710 shown in the intraoperative image data. For example, the control system may receive one or more user inputs adjusting the size and/or orientation of the target 710 to generate an updated target 715. Additionally or alternatively, the control system may automatically adjust the size and/or orientation of the target 710 to generate the updated target 715. In some examples, the control system may receive a user input selecting one or more adjustment icons 711. The user may interact with the GUI 700 by touching and dragging the adjustment icon(s) 711 around the touchscreen of the display system. Adjusting the position of the adjustment icon(s) correspondingly adjusts the size and/or orientation of the target 710 to generate the updated target 715. As discussed above, the user can scroll through the slices of intraoperative image data to determine if the updated target 715 encompasses the image of the target 710 shown in the intraoperative image data. Additionally or alternatively, the control system may determine if the updated target 715 encompasses the image of the target 710 shown in the intraoperative image data by analyzing each slice of the intraoperative image data as the user scrolls through the slices.

The size and/or orientation of the margin 712 may be automatically adjusted by the control system when the adjustment icons 711 are moved to update the size and/or orientation of the target 710. Additionally or alternatively, the size and/or orientation of the margin 712 may be adjusted when the control system receives a user input adjusting one or more adjustment icons (not shown) associated with the margin 712.

With reference to process 228 of FIG. 2, a graphical indicator 720 may be overlaid on the intraoperative image data and displayed in one or more of the view windows 702, 704, 706. The graphical indicator 720 illustrates a target position in the patient anatomy of a distal tip of a medical instrument (e.g., an ablation probe, a biopsy needle, an ultrasound transducer, etc.). As discussed above, the medical instrument may be inserted through an elongate device 730 (e.g., the elongate device 104). The graphical indicator 720 may be overlaid on the intraoperative image data prior to deployment of the medical instrument from the elongate device 730. The graphical indicator 720 may assist the operator in determining where to position the distal tip of the elongate device 730 near the target 710 by allowing the operator to move the graphical indicator 720 to different positions. The graphical indicator 720 may additionally or alternatively illustrate a target position in the patient anatomy of another portion of the medical instrument (e.g., an ablation probe, a biopsy needle, an ultrasound transducer, etc.), such as a midpoint of the medical instrument, a position on the medical instrument where one or more fiducials are located, a position on the medical instrument where one or more imagers (e.g., an ultrasound transducer or other imager) are located, a position on the medical instrument where one or more electrodes (e.g., microwave ablation electrode, electroporation electrode, etc.) are located, and/or any other portion of the medical instrument.

The location of the graphical indicator 720 in the intraoperative image data may be adjusted. In some examples, the control system may receive a user input selecting the graphical indicator 720 on the GUI 700 via a touchscreen of the display system. The user input may include touching and dragging the graphical indicator to a new location in the intraoperative image data. Any other user input, such as clicking a mouse and dragging the cursor to a new location, may be used to update the location of the graphical indicator 720. The location of the graphical indicator 720 may be adjusted to fine tune the target position of the distal tip of the medical instrument based on the position and/or orientation of the target 710 and any other anatomical structures or critical structures shown in the intraoperative image data.

In some examples, the graphical indicator 720 may be out of the view that is displayed in the view window 706. In such examples, an icon or other indicator (e.g., textual, audible, haptic, etc.) may indicate where the graphical indicator 720 is located. In some examples, the target 710 may be out of the view that is displayed in the view window 706. In such examples, an icon or other indicator (e.g., textual, audible, haptic, etc.) may indicate where the target 710 is located.

In examples when the medical instrument is an ablation probe, the treatment zone 740 may be an ablation zone. The treatment zone 740 may be overlaid on the intraoperative image data and displayed in one or more of the view windows 702, 704, 706. In some examples, when the location of the graphical indicator 720 is adjusted, the size and/or orientation of the treatment zone 740 is also adjusted.

The treatment zone 740 represents the predicted area that will be treated (e.g., ablated) by the medical instrument, such as an ablation probe, during a single treatment procedure, such as a single delivery of energy for an uninterrupted duration of time during an ablation procedure. The area covered by the treatment zone 740 may be an ablation region. An additional uninterrupted delivery of energy at a different time and/or a different location can create an additional separate treatment zone covering a separate treatment region (e.g., a separate ablation region). In some cases, separate treatment zones and treatment regions may be used, as will be described in more detail below.

The predicted size and shape of the treatment zone 740 is based on the design construction of the ablation probe, an amount of energy applied to the probe, a duration of time the energy is applied to the probe, and one or more tissue characteristics of the target 710 within which the ablation probe is deployed. The tissue characteristics of the target 710 may include density, hardness, an emphysema percentage surrounding the target 710, fibrosis, necrotic tissue, proximity to critical structures, or any other physical characteristic of the target 710 or of the anatomy surrounding the target 710. Accordingly, ablation zones of various shapes and sizes may be predicted by altering the duration of power and energy delivery at different ablation probe transducer locations within the anatomy with different tissue characteristics.

In some examples, the treatment zone 740 may be determined during a preoperative planning procedure. The preoperatively determined treatment zone may be overlaid on the intraoperative image data and displayed in one or more of the view windows 702, 704, 706. The size/orientation of the treatment zone 740 may be adjusted as needed to account for any differences in the patient anatomy (e.g., size and/or position of anatomical structures, target, etc.) that may have occurred since the size/orientation of the preoperative treatment zone was determined.

In some examples, based on the real-time position and orientation of the medical instrument in the intraoperative imaging data, treatment parameters for the treatment to be applied by the medical instrument may be adjusted. For example, when the medical instrument is an ablation probe, the size/orientation of the treatment zone 740 may be adjusted by altering the power settings, duration settings, or location of the ablation probe, which may alter the center of the treatment zone 740. The treatment parameters may be adjusted by the control system and/or by the user.

The treatment zone 740 may include one or more margins, such as a margin 742 and a margin 744. One or both of the margin 742 and the margin 744 may be overlaid on the intraoperative image data and displayed in one or more of the view windows 702, 704, 706. The margin 742 is positioned between the treatment zone 740 and the margin 712 of the target 710. The margin 744 surrounds the treatment zone 740. The margin 744 may indicate a safety margin to prevent a treatment (e.g., ablation energy) from impacting critical structures or other anatomical structures that may be in close proximity to the target 710. The critical structures may be in close proximity to the target 710 when the critical structures are within a threshold distance from the target 710 or a threshold distance from the treatment zone 740. Additional details regarding the treatment zone 740 will be discussed below. The margin 742 may indicate a buffer zone to ensure that the target 710 is fully treated by the medical instrument. For example, because the treatment zone 740 surrounds the margin 742, and because the margin 742 surrounds the margin 712 of the target 710, the control system and/or the user can determine that the ablation treatment will fully treat the target 710.

In some examples, the margin 742 and the margin 712 represent the same margin. The margin 742 is calculated as a distance measured radially inward from the treatment zone 740. The margin 712 is calculated as a distance measured radially outward from the target 710 and/or the updated target 715. Both the margin 742 and the margin 712 indicate a buffer zone to ensure that the target 710 is fully treated by the medical instrument. One or both of the margin 742 and the margin 712 may be displayed in the GUI 700.

In some examples, more than one ablation or other treatment may be needed to fully treat the target 710. In such examples, one or more additional graphical indicators may be overlaid on the intraoperative image data, and one or more additional treatment zones corresponding to the additional graphical indicator(s) may be overlaid on the intraoperative image data as well.

As discussed above, the location of the graphical indicator 720 may be adjusted. When the location of the graphical indicator 720 is adjusted, the size and/or orientation of the treatment zone 740 may also be adjusted. For example, the treatment zone 740 may move with the graphical indicator 720 as the location of the graphical indicator 720 is adjusted. For example, FIG. 7C shows the graphical indicator 720 in an initial location, and FIG. 7D shows the graphical indicator 720 in an updated location. As seen in FIG. 7D, the treatment zone 740 has been adjusted based on the updated location of the graphical indicator 720.

In some examples, the medical instrument may be deployed from the elongate device 730 before or after the location of the graphical indicator 720 is finalized. FIG. 7E illustrates a view window 704, which may be the virtual camera view 604 in the GUI 600 discussed above. In some examples, one, some, or all of the features discussed below that may be displayed in the view window 704 may be hidden from view. For example, a user input may be received indicating that one or more particular features should be hidden from view in the view window 704.

In FIG. 7E, the view window includes the graphical indicator 720, which has been transformed to be in the elongate device reference frame 350, as discussed above. The view window 704 includes an image of the graphical indicator 720, an image of the updated target 715, an icon 752, which may be a “distance to target” icon, and an icon 754, which may be a “distance to graphical indicator” icon. The “distance to the target” icon 752 illustrates a distance 751 between the distal tip (or other reference portion) of the elongate device 730 and a near edge (e.g., a closest edge) of the updated target 715. The distance 751 is shown in FIG. 7E to be 10 mm. This distance is an illustrative distance only, and the distance 751 may change as the elongate device 730 is moved relative to the target 715. The “distance to the target” icon 752 may also illustrate a distance 753 between the distal tip (or other reference portion) of the elongate device 730 and a far edge (e.g., a farthest edge) of the updated target 715. The distance 753 is shown in FIG. 7E to be 15 mm. This distance is an illustrative distance only, and the distance 753 may change as the elongate device 730 is moved relative to the target 715. In some examples, the view window 704 may include a virtual trajectory icon, which may represent a trajectory that was planned using preoperative imaging data. The “distance to the target” icon 752 may additionally or alternatively illustrate one or more of a distance between the distal tip (or other reference portion) of the elongate device 730 and a near edge (e.g., a nearest edge) of the updated target 715, a distance between the distal tip (or other reference portion) of the elongate device 730 and a far edge of the treatment zone 740, a distance between the distal tip (or other reference portion) of the elongate device 730 and a near edge of the treatment zone 740, a distance between the distal tip (or other reference portion) of the elongate device 730 and a center of the updated target 715, or a distance between the distal tip (or other reference portion) of the elongate device 730 and any designated point within the updated target 715 or the treatment zone 740.

The “distance to the graphical indicator” icon 754 illustrates a distance 755 between the distal tip (or other reference portion) of the elongate device 730 and the graphical indicator 720. The distance 755 is shown in FIG. 7E to be 21 mm. This distance is an illustrative distance only, and the distance 755 may change as the elongate device 730 is moved relative to the target 715. In some examples where the graphical indicator 720 has a three-dimensional shape (e.g., a sphere or ellipsoid), the “distance to the graphical indicator” icon 754 may illustrate a distance between the distal tip (or other reference portion) of the elongate device 730 and a near edge (e.g., a nearest edge) of the graphical indicator 720 and/or a distance between the distal tip (or other reference portion) of the elongate device 730 and a far edge (e.g., a farthest edge) of the graphical indicator 720 in addition to or instead of the distance 755. For example, the “distance to the graphical indicator” icon 754 may illustrate a distance 756 between the distal tip (or other reference portion) of the elongate device 730 and a near edge (e.g., a closest edge) of the graphical indicator 720. The distance 756 is shown in FIG. 7E to be 11 mm. This distance is an illustrative distance only, and the distance 756 may change as the elongate device 730 is moved relative to the graphical indicator 720. The “distance to the graphical indicator” icon 754 may also illustrate a distance 757 between the distal tip (or other reference portion) of the elongate device 730 and a far edge (e.g., a farthest edge) of the graphical indicator 720. The distance 757 is shown in FIG. 7E to be 31 mm. This distance is an illustrative distance only, and the distance 757 may change as the elongate device 730 is moved relative to the graphical indicator 720.

Based on the registration between the intraoperative image reference frame 450 and the elongate device reference frame 350, the graphical indicator 720 may be transformed from the intraoperative image reference frame 450 to the elongate device reference frame 350. Additionally or alternatively, any other critical structures may be transformed from the intraoperative image reference frame 450 to the elongate device reference frame 350. The transformed graphical indicator 720 and/or the transformed critical structures may be displayed in the GUI 600 during navigation of the elongate device 730 toward the target 710. Being able to view the graphical indicator 720 and/or the critical structures in the elongate device reference frame 350 may assist with the navigation of the elongate device 730 to its target location in the patient anatomy.

FIG. 7F illustrates a menu 780 and a view window 708 of the GUI 700. As discussed above, in addition to indicating an ablation zone, the treatment zone 740 may be a reference area that may additionally or alternatively indicate a radiation zone, a fiducial zone, an area where tissue and other anatomic structures may be affected by any other treatment or procedure, or an area of focus within or surrounding an anatomic structure, such as the target 715. As also discussed above, the treatment zone 740 and any other reference area may be determined during the preoperative planning procedure. The preoperatively determined treatment zone/reference area(s) may be overlaid on the intraoperative image data and displayed in one or more of the view windows 702, 704, 706, 708. The size/orientation of the treatment zone/reference area(s) may be adjusted as needed to account for any differences in the patient anatomy (e.g., size and/or position of anatomical structures, target, etc.) that may have occurred since the size/orientation of the preoperative treatment zone/reference area(s) was determined. Additionally, a margin may be added to one or more of the reference areas or the target during the preoperative planning procedure. The preoperatively determined margin(s) may be overlaid on the intraoperative image data and displayed in one or more of the view windows 702, 704, 706, 708. The size/orientation of the margin(s) may be adjusted as needed to account for any differences in the patient anatomy (e.g., size and/or position of anatomical structures, target, etc.) that may have occurred since the size/orientation of the preoperative margin(s) was determined.

The menu 780 may include one or more of a “direct placement” icon 782, a “trajectory placement” icon 784, and/or a “margin” icon 786. The GUI 700 may include one or more view icons, such as an axial view icon 791, a coronal view icon 792, a sagittal view icon 793, a tip view icon 794, and/or a side view icon 795. The tip view and the side view may each display the patient anatomy in the view window 708 from respective oblique views. The oblique views may be views of the patient anatomy that are displayed based on a view direction relative to the elongate device 730. For example, the tip view displays the patient anatomy as shown from a perspective that includes a view direction aligned with a longitudinal axis of the distal end 732 of the elongate device 730. The side view displays the patient anatomy as shown from a perspective that illustrates a side of the elongate device 730 and includes a view direction that is generally normal to the longitudinal axis of the elongate device 730. The oblique views may additionally or alternatively be based on a trajectory between two points, such as a trajectory between the distal tip 732 of the elongate device 730 and a second point at any location on the elongate device 730. The oblique views may additionally or alternatively be based on a trajectory between two points, such as a trajectory between the distal tip 732 of the elongate device 730 and any other second point (e.g., the center of the treatment zone 740, the center of the updated target 715, a fiducial, etc.). The tip view and the side view, for example, may represent planes extending between the distal tip 732 and the second point. In some examples, the second point may be selected based on a received user input. In contrast, the axial view, coronal view, and sagittal view illustrate views of the patient anatomy that are displayed based on a view direction relative to the patient.

A user input may be received at the GUI 700 to indicate which one or more reference areas determined during the preoperative planning procedure and/or new reference areas will be overlaid on the intraoperative imaging data displayed in the view window 708. The reference areas may be placed directly over the intraoperative imaging data (e.g., by receiving a user input at the “direct placement” icon 782) and/or may be placed over the intraoperative imaging data with a trajectory illustrating the spatial relationship between the reference area and the distal end 732 of the elongate device 730 (e.g., by receiving a user input at the “trajectory placement” icon 784). One, some, or all of the reference areas and/or the target 715 may include a margin. The margin may be displayed and adjusted by receiving a user input at the margin icon 786.

FIG. 7G illustrates the view window 708 from the coronal view with a reference area 850 displayed. The reference area 850 is a direct placement reference area. For example, the reference area 850 is displayed in the view window 708 without any illustrated spatial relationship between the reference area and the distal end 732 of the elongate device 730. The reference area 850 may indicate an ablation zone, a radiation zone, a fiducial zone, an area where tissue and other anatomic structures may be affected by any other treatment or procedure, or an area of focus near the target 715. In some examples, the size and/or orientation of the reference area 850 may be adjusted. For example, the control system may receive one or more user inputs adjusting the size and/or orientation of the reference area 850 to generate an updated reference area. Additionally or alternatively, the control system may automatically adjust the size and/or orientation of the reference area 850 to generate the updated reference area. In some examples, the control system may receive a user input selecting one or more adjustment icons 852. The user may interact with the GUI 700 by touching and dragging the adjustment icon(s) 852 around the touchscreen of the display system. Adjusting the position of the adjustment icon(s) 852 correspondingly adjusts the size and/or orientation of the reference area 850 to generate the reference area. As discussed above, the user can scroll through the slices of intraoperative image data to determine if the updated reference area encompasses the portions of the patient anatomy shown in the intraoperative image data that the user intends for the reference area 850 to cover. Before or after the size/orientation of the reference area 850 has been adjusted, a user input may be received at a “place” icon 788 to finalize the overlaid location of the reference area 850 on the intraoperative imaging data in the view window 708.

FIG. 7H illustrates the view window 708 from the side view with the reference area 850 and a reference area 860 displayed. The reference area 860 is a trajectory placement reference area. For example, the reference area 860 is displayed in the view window 708 with a trajectory marker 862 illustrating a spatial relationship between the reference area 860 and the distal end 732 of the elongate device 730. For example, the trajectory marker 862, which may be a solid line, a dotted line, a dashed line, or any other symbol, extends between the distal end 732 of the elongate device 730 and a center of the reference area 860.

The reference area 860 may indicate an ablation zone, a radiation zone, a fiducial zone, an area where tissue and other anatomic structures may be affected by any other treatment or procedure, or an area of focus near the target 715. In some examples, the size and/or orientation of the reference area 860 may be adjusted in the same manner described above for adjusting the size/orientation of the reference area 850. For example, the diameter, length, far edge offset, and other characteristics of the reference area 860 may be adjusted via user inputs received at an adjustment menu 865. The size and/or orientation of the reference area 850 may similarly be adjusted using the same or a different adjustment menu as the adjustment menu 865. Before or after the size/orientation of the reference area 860 has been adjusted, a user input may be received at the “place” icon 788 to finalize the overlaid location of the reference area 860 on the intraoperative imaging data in the view window 708.

As also shown in FIG. 7H, the reference area 860 includes a margin 864. As discussed above with respect to the treatment zone 740, the margin 864 of the reference area 860 may extend radially inward and/or radially outward from the reference area 860. FIG. 7H shows the margin 864 extending both radially inward and radially outward from the reference area 860. In some examples, the size and/or orientation of the margin 864 may be adjusted in the same manner described above for adjusting the size/orientation of the reference area 860.

FIG. 7I illustrates the view window 708 from the side view with the reference area 850, the reference area 860, and the margin 712 of the target 715 displayed. As discussed above with respect to FIG. 7B, the size/orientation margin 712 may be adjusted. One or more of the margins associated with each of the reference area 850, the reference area 860, and the target 715 may be hidden from view. For example, in FIG. 7I, the margins for the reference area 850 and the reference area 860 are hidden, and the margin 712 for the target 715 is displayed.

With reference to process 232 of FIG. 2, after the medical instrument is deployed, an intraoperative imaging scan may be performed while the medical instrument is deployed. Based on the intraoperative imaging data captured during this intraoperative imaging scan, the real-time position of the treatment zone 740 may be determined. The control system may automatically adjust the size and/or orientation of the treatment zone to avoid any critical structures and/or to fully surround the target based on the updated intraoperative imaging data. Additionally or alternatively, the control system may receive a user input adjusting the size and/or orientation of the treatment zone.

With reference to FIG. 8A, the GUI 700 may include an intraoperative external image 760 and an icon menu 765. The intraoperative external image 760 may be received at a control system from an intraoperative external imaging system. In some examples, the intraoperative external imaging system may be a cone beam CT (“CBCT”) imaging system than generates intraoperative CBCT image data, although any suitable imaging technique, such as conventional CT or fluoroscopy techniques, may be used without departing from the examples of the present disclosure. The intraoperative external imaging data may be received at a control system or other processing platform associated with the elongate device 730. It is also contemplated that in some examples the shape data associated with the elongate device 730 may be transferred to the imaging system, or both the shape data and the intraoperative external imaging data may be transferred to a common platform for processing. In this regard, registration of the shape data of the elongate device 730 to the intraoperative external imaging data may be performed by the control system, by the imaging system, or by another platform in operable communication with the intraoperative external imaging system and the control system. In some examples, receiving the intraoperative external imaging data may include receiving one or more timestamps associated with the intraoperative external imaging data. A first timestamp may indicate the start time of the intraoperative external imaging and a second timestamp may additionally indicate a stop time of the intraoperative external imaging. Alternatively, a timestamp may be associated with each instance of intraoperative external imaging data. In order to ensure accurate correlation, a clock of the control system of the elongate device 730 may be synchronized with a clock of the intraoperative external imaging system, and each instance of shape data may also be associated with a timestamp. In this regard, each timestamped instance of intraoperative external imaging data may be paired with a correspondingly timestamped instance of shape data.

As discussed above, the intraoperative external image 760 may include an image of the elongate device 730 and the medical instrument 770 (e.g., an ablation probe) when the medical instrument 770 is deployed from the elongate device 730. In some examples, the position and/or orientation of the medical instrument 770 may be adjusted.

As shown in FIG. 8B, the GUI 700 may include an increase icon 802 and a decrease icon 804. An insertion distance D1 of the medical instrument 770, which may be illustrated by an insertion distance icon 800, may increase when the control system receives a user input selecting the increase icon 802. Similarly, the insertion distance D1 may decrease when the control system receives a user input selecting the decrease icon 804. The insertion distance D1 may be increased or decreased in increments of 1 mm but may be increased or decreased in increments of 0.5 mm, 2 mm, 3 mm, or any other desired distance. To reset the insertion distance D1 to a default insertion distance, an icon 806 may be selected. In some examples, the medical instrument 770 may be hidden and removed from the virtual navigation view 602. Any adjustments made to the insertion distance D1 and/or any other adjustments made to the medical instrument 770 may be confirmed when the control system receives a user input selecting a “Done” icon 808. The confirmed adjustments may be revisited and further adjusted at any time.

In some examples, the control system may receive a user input selecting (e.g., touching or clicking) a distal end 772 of the medical instrument 770 and the distal end 732 of the elongate device 730 to measure the insertion distance D1 of the medical instrument 770. Additionally or alternatively, the control system may determine the insertion distance D1 of the medical instrument 770 using imaging analysis. The insertion distance may be adjusted to align the medical instrument 770 with the treatment zone 740. This may result in more efficient and more accurate treatment of the target structure 715.

As shown in FIG. 8C, the medical instrument 770 is shown as bending away from a longitudinal axis A of the elongate device 730 by an angle 810. The angle 810 may be measured from the longitudinal axis A of the elongate device 730 to the medical instrument 770. The degree of the angle 810 may be shown in an icon 812 of the GUI 700. The GUI 700 may further include an increase icon 814 and a decrease icon 816. The angle 810 may increase when the control system receives a user input selecting the increase icon 814. Similarly, the angle 810 may decrease when the control system receives a user input selecting the decrease icon 816. The angle 810 may be increased or decreased in increments of 1° (i.e., one degree) but may be increased or decreased in increments of 0.5°, 2°, 3°, or any other desired amount of degrees. As shown in FIG. 8C, the angle 810 may be 30°. In some examples, the medical instrument 770 may bend to any angle within a deployment range of the elongate device 730. The angle 810 may be adjusted to align the medical instrument 770 with the treatment zone 740. This may result in more efficient and more accurate treatment of the target structure 715.

With reference to process 234 of FIG. 2, during or after the confirmation of the treatment zone 740 and/or the confirmation of the position/orientation of the medical instrument, the elongate device 730 may be retracted. The elongate device 730 may be retracted to prevent the elongate device from being damaged during the treatment procedure (e.g., an ablation procedure) performed by the medical instrument (e.g., an ablation probe). In some examples, the control system may receive a user input instructing the elongate device 730 to retract.

The distance 755 between the distal tip of the elongate device 730 and the graphical indicator 720 shown in FIG. 7E may be used to confirm that the elongate device 730 has been retracted. For example, the distance 755 may change from 21 mm to 24 mm to indicate that the elongate device 730 has been retracted 3 mm away from the graphical indicator 720 and therefore has been retracted 3 mm away from the distal tip of the medical instrument. Any other retraction distance may be used, such as 5 mm, 4 mm, 2 mm, etc. The desired retraction distance may vary based on the type of treatment performed by the medical instrument, the strength of the treatment, or any other similar factor.

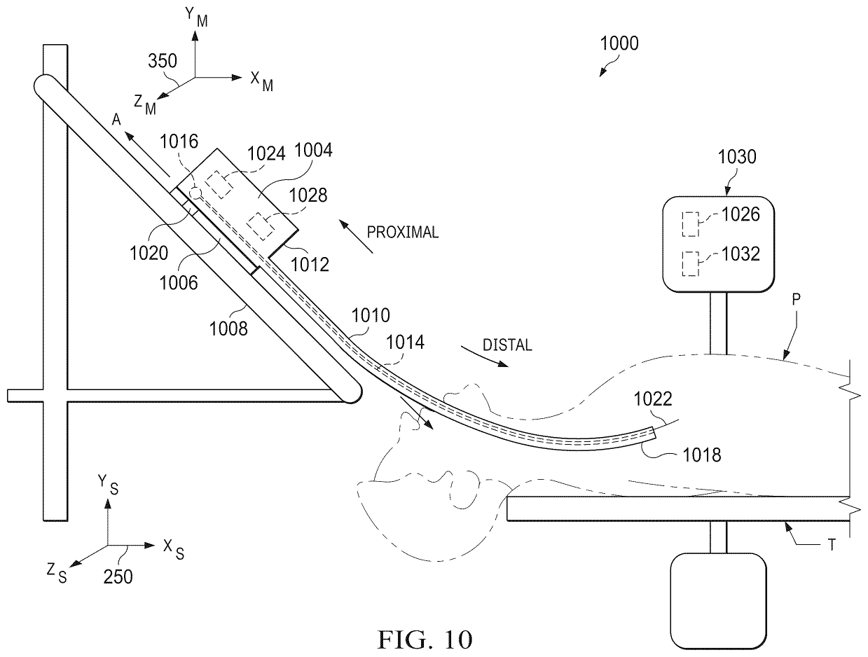

In some examples, the registration techniques of this disclosure, such as those discussed in relation to processes 210 and 222 of FIG. 2, may be used in an image-guided medical procedure performed with a robot-assisted medical system as shown in FIGS. 9 and 10. FIG. 9 illustrates a clinical system 10 includes a robot-assisted medical system 900 and an intraoperative imaging system 918. The robot-assisted medical system 900 generally includes a manipulator assembly 902 for operating a medical instrument system 904 (including, for example, elongate device 104) in performing various procedures on a patient P positioned on a table T in a surgical environment 901. The manipulator assembly 902 may be robot-assisted, non-assisted, or a hybrid robot-assisted and non-assisted assembly with select degrees of freedom of motion that may be motorized and/or robot-assisted and select degrees of freedom of motion that may be non-motorized and/or non-assisted. A master assembly 906, which may be inside or outside of the surgical environment 901, generally includes one or more control devices for controlling manipulator assembly 902. Manipulator assembly 902 supports medical instrument system 904 and may optionally include a plurality of actuators or motors that drive inputs on medical instrument system 904 in response to commands from a control system 912. The actuators may optionally include drive systems that when coupled to medical instrument system 904 may advance medical instrument system 904 into a naturally or surgically created anatomic orifice. Other drive systems may move the distal end of medical instrument system 904 in multiple degrees of freedom, which may include three degrees of linear motion (e.g., linear motion along the X, Y, Z Cartesian axes) and in three degrees of rotational motion (e.g., rotation about the X, Y, Z Cartesian axes). Additionally, the actuators can be used to actuate an articulable end effector of medical instrument system 904 for grasping tissue in the jaws of a biopsy device and/or the like.

Robot-assisted medical system 900 also includes a display system 910 (which may the same as display system 100) for displaying an image or representation of the surgical site and medical instrument system 904 generated by a sensor system 908 and/or an endoscopic imaging system 909. Display system 910 and master assembly 906 may be oriented so operator O can control medical instrument system 904 and master assembly 906 with the perception of telepresence.

In some examples, medical instrument system 904 may include components for use in surgery, biopsy, ablation, illumination, irrigation, or suction. Optionally medical instrument system 904, together with sensor system 908 may be used to gather (e.g., measure) a set of data points corresponding to locations within anatomical passageways of a patient, such as patient P. In some examples, medical instrument system 904 may include components of the imaging system 909, which may include an imaging scope assembly or imaging instrument that records a concurrent or real-time image of a surgical site and provides the image to the operator or operator O through the display system 910. The concurrent image may be, for example, a two or three-dimensional image captured by an imaging instrument positioned within the surgical site. In some examples, the imaging system components that may be integrally or removably coupled to medical instrument system 904. However, in some examples, a separate endoscope, attached to a separate manipulator assembly may be used with medical instrument system 904 to image the surgical site. The imaging system 909 may be implemented as hardware, firmware, software or a combination thereof which interact with or are otherwise executed by one or more computer processors, which may include the processors of the control system 912.

The sensor system 908 may include a position/location sensor system (e.g., an electromagnetic (EM) sensor system) and/or a shape sensor system for determining the position, orientation, speed, velocity, pose, and/or shape of the medical instrument system 904.

Robot-assisted medical system 900 may also include control system 912. Control system 912 includes at least one memory 916 and at least one computer processor 914 for effecting control between medical instrument system 904, master assembly 906, sensor system 908, endoscopic imaging system 909, and display system 910. Control system 912 also includes programmed instructions (e.g., a non-transitory machine-readable medium storing the instructions) to implement some or all of the methods described in accordance with aspects disclosed herein, including instructions for providing information to display system 910.

Control system 912 may optionally further include a virtual visualization system to provide navigation assistance to operator O when controlling medical instrument system 904 during an image-guided surgical procedure. Virtual navigation using the virtual visualization system may be based upon reference to an acquired preoperative or intraoperative dataset of anatomical passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like.

An intraoperative imaging system 918 may be arranged in the surgical environment 901 near the patient P to obtain images of the patient P during a medical procedure. The intraoperative imaging system 918 may provide real-time or near real-time images of the patient P. In some examples, the intraoperative imaging system 918 may be a mobile C-arm cone-beam CT imaging system for generating three-dimensional images. For example, the intraoperative imaging system 918 may be a DynaCT imaging system from Siemens Corporation of Washington, D.C., or other suitable imaging system. In other examples, the imaging system may use other imaging technologies including CT, MRI, fluoroscopy, thermography, ultrasound, optical coherence tomography (OCT), thermal imaging, impedance imaging, laser imaging, nanotube X-ray imaging, and/or the like.

FIG. 10 illustrates a surgical environment 1000 with a surgical reference frame (X_{S}, Y_{S}, Z_{S}) 250 in which the patient P is positioned on the table T. Patient P may be stationary within the surgical environment in the sense that gross patient movement is limited by sedation, restraint, and/or other means. Cyclic anatomic motion including respiration and cardiac motion of patient P may continue unless the patient is asked to hold his or her breath to temporarily suspend respiratory motion. Within surgical environment 1000, a medical instrument 1004 (e.g., the medical instrument system 904), having a medical instrument reference frame (X_{M}, Y_{M}, Z_{M}) 350, is coupled to an instrument carriage 1006. In this example, medical instrument 1004 includes an elongate device 1010, such as a flexible catheter, coupled to an instrument body 1012. Instrument carriage 1006 is mounted to an insertion stage 1008 fixed within surgical environment 1000. Alternatively, insertion stage 1008 may be movable but have a known location (e.g., via a tracking sensor or other tracking device) within surgical environment 1000. In these alternatives, the medical instrument reference frame is fixed or otherwise known relative to the surgical reference frame. Instrument carriage 1006 may be a component of a robot-assisted manipulator assembly (e.g., robot-assisted manipulator assembly 1002) that couples to medical instrument 1004 to control insertion motion, such as motion along an axis A, and, optionally, motion of a distal end 1018 of the elongate device 1010 in multiple directions including yaw, pitch, and roll. Instrument carriage 1006 or insertion stage 1008 may include actuators, such as servomotors, (not shown) that control motion of instrument carriage 1006 along insertion stage 1008.

In this example, a sensor system (e.g., sensor system 908) includes a shape sensor 1014. Shape sensor 1014 may include an optical fiber extending within and aligned with elongate device 1010. In one example, the optical fiber has a diameter of approximately 200 μm. In other examples, the dimensions may be larger or smaller. The optical fiber of shape sensor 1014 forms a fiber optic bend sensor for determining the shape of the elongate device 1010. In one alternative, optical fibers including Fiber Bragg Gratings (FBGs) are used to provide strain measurements in structures in one or more dimensions. Various systems and methods for monitoring the shape and relative position of an optical fiber in three dimensions are described in U.S. patent application Ser. No. 11/180,389 (filed Jul. 13, 2005) (disclosing “Fiber optic position and shape sensing device and method relating thereto”); U.S. patent application Ser. No. 12/047,056 (filed on Jul. 16, 2004) (disclosing “Fiber-optic shape and relative position sensing”); and U.S. Pat. No. 6,389,187 (filed on Jun. 17, 1998) (disclosing “Optical Fiber Bend Sensor”), which are all incorporated by reference herein in their entireties. Sensors in some examples may employ other suitable strain sensing techniques, such as Rayleigh scattering, Raman scattering, Brillouin scattering, and Fluorescence scattering. In some examples, the shape of the catheter may be determined using other techniques. For example, a history of the distal end pose of elongate device 1010 can be used to reconstruct the shape of elongate device 1010 over the interval of time.

As shown in FIG. 10, instrument body 1012 is coupled and fixed relative to instrument carriage 1006. In some examples, the optical fiber shape sensor 1014 is fixed at a proximal point 1016 on instrument body 1012. In some examples, proximal point 1016 of optical fiber shape sensor 1014 may be movable along with instrument body 1012 but the location of proximal point 1016 may be known (e.g., via a tracking sensor or other tracking device). Shape sensor 1014 measures a shape from proximal point 1016 to another point such as distal end 1018 of elongate device 1010 in the medical instrument reference frame (X_{M}, Y_{M}, Z_{M}).

Elongate device 1010 includes a channel (not shown) sized and shaped to receive a medical instrument 1022. In some examples, medical instrument 1022 may be used for procedures such as surgery, biopsy, ablation, illumination, irrigation, or suction. Medical instrument 1022 can be deployed through elongate device 1010 and used at a target location within the anatomy. Medical instrument 1022 may include, for example, image capture probes, biopsy instruments, laser ablation fibers, and/or other surgical, diagnostic, or therapeutic tools. Medical instrument 1022 may be advanced from the distal end 1018 of the elongate device 1010 to perform the procedure and then retracted back into the channel when the procedure is complete. Medical instrument 1022 may be removed from proximal end of elongate device 1010 or from another optional instrument port (not shown) along elongate device 1010.

Elongate device 1010 may also house cables, linkages, or other steering controls (not shown) to controllably bend distal end 1018. In some examples, at least four cables are used to provide independent “up-down” steering to control a pitch of distal end 1018 and “left-right” steering to control a yaw of distal end 1018.

A position measuring device 1020 provides information about the position of instrument body 1012 as it moves on insertion stage 1008 along an insertion axis A. Position measuring device 1020 may include resolvers, encoders, potentiometers, and/or other sensors that determine the rotation and/or orientation of the actuators controlling the motion of instrument carriage 1006 and consequently the motion of instrument body 1012. In some examples, insertion stage 1008 is linear, while in other examples, the insertion stage 1008 may be curved or have a combination of curved and linear sections.

An intraoperative imaging system 1030 (e.g., imaging system 918) is arranged near the patient P to obtain three-dimensional images of the patient while the elongate device 1010 is extended within the patient. The intraoperative imaging system 1030 may provide real-time or near real-time images of the patient P. One or more fiducial markers (not shown) may be positioned on the patient P during preoperative imaging and intraoperative imaging to improve registration.

In some examples, the medical instrument 1004 or another component of a robot-assisted medical system registered to the medical instrument 1004 may include an instrument clock 1024. The imaging system 1030 may include an imaging clock 1026. The clocks 1024, 1026 may be time synchronized on a predetermined schedule or in response to a synchronization initiation event generated by a user, a control system, or a synchronization system. In some examples, the clocks 1024, 1026 may be components of a synchronization system that may be a centralized or distributed system further comprising servers, wired or wireless communication networks, communication devices, or other components for executing synchronization algorithms and protocols. In some examples, the medical instrument 1004 or another component of a robot-assisted medical system registered to the medical instrument 1004 may include a communication device 1028. The imaging system 1030 may include a communication device 1032.

The singular forms “a”, “an”, and “the” are intended to include the plural forms as well, unless the context indicates otherwise. And the terms “comprises,” “comprising,” “includes,” “has,” and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components. Components described as coupled may be directly or indirectly communicatively coupled. The auxiliary verb “may” likewise implies that a feature, step, operation, element, or component is optional.

In the description, specific details have been set forth describing some examples. Numerous specific details are set forth in order to provide a thorough understanding of the examples. It will be apparent, however, to one skilled in the art that some examples may be practiced without some or all of these specific details. The specific examples disclosed herein are meant to be illustrative but not limiting. One skilled in the art may realize other elements that, although not specifically described here, are within the scope and the spirit of this disclosure.

Elements described in detail with reference to one example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in the following description, one or more elements shown and described in association with one example, implementation, or application may be incorporated into other examples, implementations, or aspects unless specifically described otherwise, unless the one or more elements would make an example or implementation non-functional, or unless two or more of the elements provide conflicting functions.

Any alterations and further modifications to the described devices, instruments, methods, and any further application of the principles of the present disclosure are fully contemplated as would normally occur to one skilled in the art to which the disclosure relates. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative example can be used or omitted as applicable from other illustrative examples. For the sake of brevity, the numerous iterations of these combinations will not be described separately. For simplicity, in some instances the same reference numbers are used throughout the drawings to refer to the same or like parts.

The systems and methods described herein may be suited for navigation and treatment of anatomic tissues, via natural or surgically created connected passageways, in any of a variety of anatomic systems, including the lung, colon, the intestines, the kidneys and kidney calices, the brain, the heart, the circulatory system including vasculature, and/or the like. Although some of the examples described herein refer to surgical procedures or instruments, or medical procedures and medical instruments, the techniques disclosed apply to non-medical procedures and non-medical instruments. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

Further, although some of the examples presented in this disclosure discuss robotic-assisted systems or remotely operable systems, the techniques disclosed are also applicable to computer-assisted systems that are directly and manually moved by operators, in part or in whole.

Additionally, one or more elements in examples of this disclosure may be implemented in software to execute on a processor of a computer system such as a control processing system. When implemented in software, the elements of the examples of the present disclosure are essentially the code segments to perform the necessary tasks. The program or code segments can be stored in a processor readable storage medium (e.g., a non-transitory storage medium) or device that may have been downloaded by way of a computer data signal embodied in a carrier wave over a transmission medium or a communication link. The processor readable storage device may include any medium that can store information including an optical medium, semiconductor medium, and magnetic medium. Processor readable storage device examples include an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc. Any of a wide variety of centralized or distributed data processing architectures may be employed. Programmed instructions may be implemented as a number of separate programs or subroutines, or they may be integrated into a number of other aspects of the systems described herein. In some examples, the control system may support wireless communication protocols such as Bluetooth, Infrared Data Association (IrDA), HomeRF, IEEE 802.11, Digital Enhanced Cordless Telecommunications (DECT), ultra-wideband (UWB), ZigBee, and Wireless Telemetry.

A computer is a machine that follows programmed instructions to perform mathematical or logical functions on input information to produce processed output information. A computer includes a logic unit that performs the mathematical or logical functions, and memory that stores the programmed instructions, the input information, and the output information. The term “computer” and similar terms, such as “processor” or “controller” or “control system”, are analogous.

Note that the processes and displays presented may not inherently be related to any particular computer or other apparatus, and various systems may be used with programs in accordance with the teachings herein. The required structure for a variety of the systems discussed above will appear as elements in the claims. In addition, the examples of the present disclosure are not described with reference to any particular programming language. It will be appreciated that a variety of programming languages may be used to implement the teachings of the present disclosure as described herein.

While certain example examples of the present disclosure have been described and shown in the accompanying drawings, it is to be understood that such examples are merely illustrative of and not restrictive to the broad disclosed concepts, and that the examples of the present disclosure not be limited to the specific constructions and arrangements shown and described, since various other modifications may occur to those ordinarily skilled in the art.

1. A system comprising:
a display system;
an elongate device;
a medical instrument configured to extend within the elongate device; and
a control system communicatively coupled to the display system, the control system configured to:
display intraoperative imaging data of a patient anatomy received from an external imaging system, wherein the intraoperative imaging data is displayed in a graphical user interface (GUI) on the display system;
display a graphical indicator overlaid on the intraoperative imaging data, wherein the graphical indicator indicates a target position of a distal tip of the medical instrument in the patient anatomy;
register the intraoperative imaging data to the elongate device;
based on the registration, transform the graphical indicator from a reference frame of the intraoperative imaging data to a reference frame of the elongate device; and
based on the target position of the distal tip of the medical instrument displayed via the graphical indicator, provide navigation guidance to guide the elongate device to a target location in the patient anatomy, wherein when the elongate device is positioned at the target location, the target position of the distal tip of the medical instrument is within a deployment range of the medical instrument.

2. The system of claim 1, wherein a portion of the intraoperative imaging data corresponds to the elongate device.

3. The system of claim 2, wherein the control system is further configured to segment the portion of the intraoperative imaging data corresponding to the elongate device.

4. The system of claim 2, wherein the registering the intraoperative imaging data to the elongate device includes comparing shape data received from the elongate device to a shape of the elongate device in the portion of the intraoperative imaging data corresponding to the elongate device.

5. The system of claim 1, wherein the control system is further configured to display a treatment zone that covers at least a portion of an anatomical target, and wherein an orientation of the treatment zone is determined based on a location of the graphical indicator in the GUI.

6. The system of claim 5, wherein the control system is further configured to receive a user input updating the location of the graphical indicator in the GUI.

7. The system of claim 6, wherein the control system is further configured to update the treatment zone as the location of the graphical indicator changes.

8. The system of claim 5, wherein the control system is further configured to display a margin surrounding the treatment zone or within the treatment zone.

9. (canceled)

10. The system of claim 5, wherein the control system is further configured to display a margin surrounding the anatomical target, wherein the treatment zone surrounds the margin.

11. The system of claim 5, wherein the control system is further configured to receive a user input adjusting one or more treatment parameters.

12. The system of claim 11, wherein the control system is further configured to update the treatment zone based on the received user input adjusting the one or more treatment parameters.

13. The system of claim 1, wherein providing navigation guidance includes determining a distance from a distal tip of the elongate device to the graphical indicator.

14. The system of claim 1, wherein providing navigation guidance includes determining a distance from a distal tip of the elongate device to an anatomical target.

15. The system of claim 1, wherein providing navigation guidance includes providing user instructions to articulate the elongate device to align a longitudinal axis of the elongate device with the graphical indicator.

16. The system of claim 15, wherein the longitudinal axis of the elongate device is aligned with a center of the graphical indicator.

17. (canceled)

18. The system of claim 1, wherein displaying the intraoperative imaging data includes displaying one or more critical structures in close proximity to an anatomical target in the patient anatomy.

19. The system of claim 1, wherein the control system is further configured to receive additional intraoperative imaging data of the patient anatomy from the external imaging system, wherein the additional intraoperative imaging data includes an image of the medical instrument extended from the elongate device.

20. The system of claim 19, wherein the control system is further configured to:
receive a user input adjusting a position of the graphical indicator; and
display the graphical indicator at an updated position based on the received user input adjusting the position of the graphical indicator.

21. (canceled)

22. The system of claim 19, wherein the control system is further configured to:
display a treatment zone that covers at least a portion of an anatomical target, wherein an orientation of the treatment zone is determined based on a location of the graphical indicator in the GUI;
receive a user input adjusting one or more treatment parameters; and
update the treatment zone based on the received user input adjusting the one or more treatment parameters.

23. (canceled)

24. The system of claim 19, wherein the control system is further configured to receive a user input adjusting a treatment zone, wherein the treatment zone covers at least a portion of an anatomical target, and wherein an orientation of the treatment zone is determined based on a location of the graphical indicator.

25-60. (canceled)
